(19) United States
(12) Patent Application Publication
(10) Pub. No.: US 2024/0423878 A2
(43) Pub. Date: Dec. 26, 2024

(54) SELF-ADHESIVE FISSURE SEALANT

(71) Applicant: KULZER GMBH, Hanau (DE)

(72) Inventors: Maria LECHMANN-DORN, Frankfurt (DE); Michael GERLACH, Hofheim (DE)

(73) Assignee: KULZER GMBH, Hanau (DE)

(21) Appl. No.: 18/284,933

(22) PCT Filed: Mar. 31, 2022
(86) PCT No.: PCT/EP2022/058711

§ 371 Date: Sep. 29, 2023

(30) Foreign Application Priority Data
Apr. 2, 2021 (DE) 10 2021 108 476.8
Apr. 2, 2021 (DE) 10 2021 108 477.6

(51) Int. Cl. A61K 6/887; A61K 6/62; A61K 6/77; A61K 6/78

(52) U.S. Cl. CPC A61K 6/887; A61K 6/54; A61K 6/62; A61K 6/77; A61K 6/78

(57) ABSTRACT

Self-adhesive, radiation-curable fissure sealants and methods comprise a white pigment, A) a monomer component comprising a) at least one difunctional urethane (meth)acrylate, b) at least one hydrophilic alkylene oxide-based difunctional (meth)acrylate-based crosslinker, c) a mixture of acidic monomers of an olefinic carboxylic acid and/or of an olefinic carboxylic anhydride in combination with an olefinic acidic monoester of a phosphoric acid and/or an olefinic acidic monoester of a thiophosphoric acid, and d) an initiator and/or an initiator system, each having an absorption maximum of 420 to 550 nm, optionally with a co-initiator, and B) a filler component comprising at least one dental glass, wherein A) the monomer component and B) the filler component are present in a mass ratio of 60:40 to 100:0 in the fissure sealant, and wherein the total composition of the fissure sealant comprises 100 wt.-%.

This application is a 371 of International Patent Application No. PCT/EP2022/058711, filed Mar. 1, 2021, which claims foreign priority benefit under 35 U.S.C. § 119 of the German Patent Application Nos. DE 10 2021 108 477.6, filed Apr. 2, 2021, and DE 10 2021 108 476.8, Apr. 2, 2021, the disclosures of which are incorporated herein by reference.

A self-adhesive, radiation-curable fissure sealant comprising A) a monomer component comprising a) at least one difunctional urethane (meth)acrylate, b) at least one hydrophilic alkylene oxide-based difunctional (meth)acrylate-based crosslinker, c) a mixture of acidic monomers of an olefinic carboxylic acid and/or of an olefinic carboxylic acid anhydride in combination with an olefinic acidic monoester of a phosphoric acid and/or an olefinic acidic monoester of a thiophosphoric acid and d) an initiator and/or an initiator system, each having an absorption maximum of 420 to 550 nm, optionally with a co-initiator and B) a filler component comprising at least one dental glass, wherein A) the monomer component and B) the filler component are present in a mass ratio of 60:40 to 100:0 in the fissure sealant, and wherein the total composition of the fissure sealant is 100 wt.-%.

Fissure sealants are preventive products to minimize the risk of caries in deep fissures or in susceptible patients. Fissure sealants are polymerizable compositions that adhere to the enamel. The standard procedure for fissure sealing involves acid etching of the enamel followed by sealing with a low viscosity flow material or glass ionomer cement-without the prior use of a bonding agent. Alternatively, suitable flow materials can be used in combination with an adhesive.

WO0025729A1 recommends special shape fillers for dental and similar materials. The material may also contain conventional glass fillers, pigments and radiopaque additives. EP363095A2 describes fluoride-delivering materials which are suggested, among other things, as underfilling materials. Example 9 describes an enamel sealing material that has 0.77 wt.-% TiO₂. A similar fluoride-releasing material is described in GB2257433A.

DIN-ISO 6874-2015 regulates a curing depth of 1.5 mm polymerization depth for fissure sealants. Since a fissure sealant usually penetrates into fissures of freshly erupted molars or premolars and is intended to provide chemical anchorage there, it is desirable that the depth of cure as well as the chemical bond to the enamel is further improved. Another challenge is that a self-adhesive fissure sealant should penetrate and chemically bond to unpretreated fissures in the enamel. Untreated fissures are understood to be fissures that have been cleaned but not previously etched.

The object of the invention was to provide a dental, self-adhesive fissure sealant which bonds adhesively to enamel with high adhesive strength. The fissure sealant to be developed should preferably be applicable without prior chemical treatment of the enamel or fissures, preferably without prior etching of the enamel or fissure surface. Furthermore, the fissure sealant should be applied without prior application of an adhesive to enamel. A further object was to provide a fissure sealant which further improves the depth of cure of the fissure sealant by means of radiation curing. The fissure sealant should also adhere to aprismatic enamel. This morphology is found in the enamel of deciduous teeth and deep pits of teeth. Furthermore, the object was to develop a self-adhesive, opaque fissure sealant with reduced water absorption (μg/mm³) and/or reduced solubility (μg/mm³). In addition, the object was to develop a Bis-GMA and HEMA free fissure sealant, since these monomers are considered to have an allergenic potential as well as a mutagenic potential.

Surprisingly, it has been possible to provide a fissure sealant which shows the same or better adhesion to the enamel after artificial aging on aprismatic enamel without a prior etching step of the enamel. Shear bond strength is determined according to DIN-ISO 29022 after artificial aging (5000 cycles of thermal cycling). A fissure sealant according to the invention shows a shear bond strength on aprismatic enamel of 14.6 MPA, while the aforementioned products with previous etching step show shear bond strengths of 0.7 to 13.9 MPa under otherwise identical conditions (Helioseal F (13.6 MPA), Clinpro (10.1 MPa), Grandio Seal (13.9 MPA), Ultra Seal XT Hydro (0.7 MPa)).

On the one hand, improved shear bond strength could be achieved by a specific combination of polymerizable acidic monomers in the fissure sealant directly with the application of the fissure sealant. Furthermore, by combining an increased content of initiator or initiator system in combination with a specific selection of reactive diluents, it was possible to adjust the viscosity and at the same time the reactivity of the fissure sealant for optimum flow of the fissure sealant with improved curing or polymerization. At the same time, the chemical bonding to the enamel surface was improved.

The objects are solved with a fissure sealant according to claim 1 and a polymerized fissure sealant according to claim 18 or 19. In the subclaims and in the description further detailed disclosures of the objects according to the invention are mentioned.

A self-adhesive fissure sealant offers the advantage of using the sealant without prior use of an adhesive system and etchant on the tooth surface comprising enamel and optionally dentin. The fissure sealant according to the invention exhibits a high adhesive strength due to the synergistic effect of the particularly coordinated monomers in the formulation and optionally in combination with the initiator system. On enamel, the fissure sealant intrinsically develops a very high bond strength due to a self-etching property. The bond strength values achieved are significantly higher than those of the competitor on the market. On untreated enamel, even after artificial aging, the fissure sealant shows a higher bonding performance than the products of market competitors, which provide for a prior etching step. While the fissure sealant according to the invention, measured according to ISO 29022 (BTE-unground, TWL, 5000 cycles), shows a shear bond strength of 16.3 [MPa] on untreated enamel compared to competitor products, with prior etching of the enamel before application, shear bond strengths in the range of 12.1 [MPa] to below 1 [MPa]. Thus, despite etching, shear bond strengths were determined for Helioseal F [12.1 MPa], Helioseal clear [11.4 MPa], Clinpro [6.7 MPa], Fissurit FX [4.2 MPa] and Grandio Seal [below 1 MPa]. The shear bond strength of the fissure sealant according to the invention was increased from the very good value of 16.3 [MPa] without etching step to 21.1 [MPa] with etching step, in each case measured after artificial aging (TWL, 5000 cycles).

An object of the invention is a self-adhesive, radiation-curable, dental fissure sealant, in particular a fissure sealant which is cureableor polymerizable with visible light, comprising
A) a monomer component comprising
   a) at least one at least difunctional urethane (meth)acrylate,
   b) at least one hydrophilic alkylene oxide-based difunctional (meth)acrylate-based crosslinker which i) does not comprise urethane (meth)acrylate and/or ii) does not comprise aromatic residues, or a mixture of at least two of these crosslinkers, and/or
   c) acidic monomers, in particular a mixture of acidic monomers, comprising
      i) at least one olefinic carboxylic acid and/or at least one olefinic carboxylic acid anhydride, in particular at least one carboxylic acid-functionalized and/or carboxylic acid anhydride-functionalized (meth)acrylate monomer, and
      ii) at least one olefinic acid ester of a phosphoric acid, in particular a monoester of a phosphoric acid and/or at least one olefinic acid ester of a thiophosphoric acid, in particular a monoester of a thiophosphoric acid, preferably at least one monoester of a phosphoric acid with a mono-, di-, tri-, or tetra-(meth)acrylate, monoester of a phosphoric acid and at least one urethane (meth)acrylate, and/or monoester of a thiophosphoric acid with a mono-, di-, tri-, or tetra-(meth)acrylate, in particular monoester of phosphoric acid of the formula I and/or II, or a mixture of i) and ii),
   d) at least one initiator and/or initiator system each having an absorption maximum of 420 to 550 nm and comprising a diketone and optionally at least one co-initiator, in particular selected from a tert-amine and a 1,2-methylenedioxybenzene, wherein in each case independently a (meth)acrylate, in particular comprising corresponding urethanes according to a) and/or b), is selected from methacrylate and acrylate, and optionally
B) a filler component comprising at least one dental glass or a mixture of dental glasses, in particular the dental glass has an average particle size d₅₀ of 0.2 to 2.0 μm (micro meter), wherein A) the monomer component and B) the filler component are present in a mass ratio of 50:50 to 100:0, preferably from 60:40 to 100:0, are present in the fissure sealant, and the total composition of the fissure sealant being 100 wt.-% (synonymus to % by weigth). Preferably, A) and B) are present in the fissure sealant as a mixture. Preferably, the fissure sealant is present as a flowable fissure sealant material which is readily visible as such and distinguishable from the tooth structure and which cures with visible light.

Thus, the aforementioned urethane (meth)acrylates also comprise urethane methacrylate and/or urethane acrylate as (meth)acrylate constituent. Accordingly, carboxylic anhydride-functionalized (meth)acrylate monomers also comprise the respective carboxylic anhydride-functionalized methacrylate and/or carboxylic anhydride-functionalized acrylate monomers. The same applies to monoesters of phosphoric acids based on (meth)acrylates, such as (meth)acryloyloxy derivatives; these may be selected from the respective methacrylates and acrylates of the respective compound. This applies throughout to all (meth)acrylates, that they are selected from methacrylates and acrylates.

In an alternative embodiment, the fissure sealant may comprise at least one white pigment that is preferably present from 0.1 to 1.0 wt.-% with respect to the total composition. Alternatively or additionally, the fissure sealant may comprise e) at least one white pigment and optionally at least one dye, photochromic compound and/or compound which changes color in the presence of free-residue compounds is present, which in particular fades again over time. In particular, oxides with a high refractive index, such as titanium dioxide, Al₂O₃, ZrO₂ or ZnO, are considered as white pigment or synonymously white pigment. Titanium dioxide is preferred. The white pigment is advantageously contained in the fissure sealant at 0.3-1.0 wt.-%.

The designation (meth)acrylate, (meth)acrylate-based or urethane (meth)acrylate disclose throughout, both acrylate-, acrylate- and/or urethane acrylate-based and preferably methacrylate, methacrylate- and/or urethane methacrylate-based. The same applies to the (meth)acrylates of monoesters of phosphoric acids.

Even though the fissure sealant is described as a fissure sealant comprising a monomer component A) and a filler component B), these two components are present in the fissure sealant as a mixture. This type of disclosure is known to the person skilled in the art in the dental field, since it merely serves to characterize the two initially prepared components A) and B) before they are mixed to form the fissure sealant. The fissure sealant is available as a mixture ready for use. The monomer component A) and filler component B) can be mixed in a ratio of 60:40 to 100:0 for the preparation of the fissure sealant, in particular 60:40 to 80:20 or 90:10 to 100:0, preferably 90:10 to 99:1, particularly preferably 95:5 to 100:0. In an alternative, filler-free fissure sealants are particularly preferred.

The initiator system preferably comprises an initiator such as a diketone and a co-initiator. Particularly preferred is at least one initiator and/or initiator system with an absorption maximum of 420 to 550 nm each at least comprising a cycloaliphatic diketone and as co-initiator an aromatic tert-amine. Preferably, the fissure sealant d) comprises at least one initiator and/or initiator system comprising
i) diketone and ii) co-initiator selected from a tert-amine present at a molar ratio of ii): i) of greater than or equal to 1.15, and in particular wherein i) the diketone is present at greater than or equal to 0.5 wt.-%, preferably greater than 0.4 wt.-% in the total composition of the dental material of 100 wt.-%. Alternatively, the diketone is present in the fissure sealant at greater than or equal to 0.15 wt.-%, preferably greater than 0.2 wt.-%, more preferably greater than or equal to 0.3 wt.-%.

In a particularly preferred embodiment, the fissure sealant comprises in the
A) monomer component d) from 0.3 to 2.5 wt.-% of at least one diketone, in particular 1,7,7-trimethyl-bicyclo-[2.2.1]-hepta-2,3-dione (champherquinone), and from 0.6 to 2.5 wt.-% of at least one tert-amine, in particular 2-n-butoxyethyl-4-(dimethylamino) benzoate relative to the total composition of 100 wt.-% of the A) monomer component. Preferably, the fissure sealant comprises from 0.3 to 2.5 wt.-% of at least one diketone selected from 1,7,7-trimethyl-bicyclo-[2.2.1]-hepta-2,3-dione (champherquinone), phenylpropanedione, and from 0.6 to 2.5 wt.-% of at least one co-initiator selected from 2-n-butoxyethyl-4-(dimethylamino) benzoate and/or piperonyl alcohol, wherein the mass ratio of diketone to co-initiator is from 1:1.5 to 1:3, preferably 1:2 with +/−0.15.

According to a preferred embodiment, a fissure sealant is comprised having a
A) Monomer component comprising
   a) 40 to 80 wt.-%, in particular 50 to 80 wt.-% of at least one at least difunctional urethane (meth)acrylate,
   b) 10 to 35 wt.-%, in particular 15 to 35 wt.-%, preferably 20 wt.-% with +/−5 wt.-%, in particular with +/−3 wt.-%, of at least one hydrophilic alkylene oxide-based difunctional (meth)acrylate-based crosslinker which i) does not comprise urethane (meth)acrylate and/or ii) does not comprise aromatic residues, or a mixture of at least two of these crosslinkers,
   c) 5 to 30 wt.-%, in particular 10 to 20 wt.-%, of a mixture of acidic monomers as disclosed,
   d) from 0.3 to 5 wt.-%, in particular from 0.3 to 3 wt.-%, of at least one initiator and/or initiator system, in each case having an absorption maximum of from 420 to 550 nm, the content of the at least one diketone being 0.3 wt.-% in the monomer component, and optionally with at least one tert-amine as co-initiator or a 1,2-methylenedioxybenzene as co-initiator, in particular with at least 0.6 wt.-% of co-initiator,
   e) 0 to 1.0 wt.-%, in particular 0.1 to 0.5 wt.-% of at least one pigment selected from titanium dioxide, and optionally dye, UV stabilizer and/or vis stabilizer, wherein the total composition of monomer component A) is 100 wt.-%, and optionally
B) a filler component comprising
   a) 90 to 100 wt.-% of at least one dental glass or a mixture of dental glasses,
   b) 0 to 10 wt.-%, in particular 5 to 10 wt.-%, of inorganic fluoride, preferably ytterbium fluoride, metal oxide, such as SiO₂, ZrO₂, and mixed oxides, in particular mixed oxides of SiO₂ and ZrO₂,
   c) 0 to 1.0 wt.-%, in particular 0.1 to 0.5 wt.-%, of at least one pigment selected from titanium dioxide, and optionally dye, UV stabilizer and/or vis stabilizer, and wherein A) the monomer component and B) the filler component are present in a mass ratio of 60:40 to 100:0 in the fissure sealant, and wherein the total composition of the fissure sealant is 100 wt.-%.

It is preferred if the content of at least one white pigment is from 0 to 1.0 wt.-%, preferably from 0.1 to 1.0 wt.-%, based on the total composition.

In the monomer component, component b) of the hydrophilic alkylene oxide-based difunctional (meth)acrylate-based crosslinker may be present in a preferred alternative as a mixture of at least two hydrophilic alkylene oxide-based di-functional (meth)acrylate-based crosslinkers.

Particularly preferably, the fissure sealant comprises in the monomer component at least
a) urethane dimethacrylate (UDMA) and at least one monomer, preferably at least two different monomers of the combinations i) and ii) or i) and iii) or i), ii) and iii) with
b) i) TEGDMA, and ii) PEGDA (polyethylene glycol diacrylate) with n=2 to 9, PPGDA (polypropylene glycol diacrylate) with n=2 to 9, PEGDMA (polyethylene glycol dimethacrylate) with n=2 to 9, PPGDMA (polypropylene glycol dimethacrylate) with n=2 to 9, or DDDMA (dodecanediol dimethacrylate), and iii) BDMA, 1,4-butanediol dimethacrylate (1,4-BDMA), or pentaerythritol tetraacrylate. Alkylene oxide-based difunctional (meth)acrylate-based crosslinkers, i.e. alkylene oxide-based difunctional methacrylate-based crosslinkers or alkylene oxide-based difunctional acrylate-based crosslinkers, are generally considered to be ethers and esters of di- to polyols with (meth)acrylic acids. (meth)acrylate is also understood here to mean methacrylate or acrylate monomers.

Further preferably, the fissure sealant comprises in the monomer component at least a) difunctional urethane (meth)acrylate, preferably urethane dimethacrylate (UDMA), and at least one monomer selected from b) PEGDA (polyethylene glycol diacrylate) with n=2 to 9, PPGDA (polypropylene glycol diacrylate) with n=2 to 9, in particular 2 to 8, PEGDMA (polyethylene glycol dimethacrylate) with n=2 to 9, PPGDMA (polypropylene glycol dimethacrylate) with n=2 to 9 or DDDMA (dodecanediol dimethacrylate), and iii) BDMA, 1,4-butanediol dimethacrylate (1,4-BDMA) or pentaerythritol tetraacrylate. As well as, in each case, the acrylates of the aforementioned compounds.

In preferred alternatives, the fissure sealant may comprise in the monomer component at least a) difunctional urethane (meth)acrylate, preferably urethane di methacrylate (UDMA), and at least one monomer, preferably at least two different monomers, selected from b) i) TEGDMA, and ii) PEGDA (polyethylene glycol diacrylate) with n=2 to 9, PPGDA (polypropylene glycol diacrylate) with n=2 to 9, PEGDMA (polyethylene glycol dimethacrylate) with n=2 to 9, PPGDMA (polypropylene glycol dimethacrylate) with n=2 to 9 or DDDMA (dodecanediol dimethacrylate), and iii) BDMA, 1,4-butanediol dimethacrylate (1,4-BDMA) or pentaerythritol tetraacrylate.

Particularly preferred fissure sealants comprise monomer component A) or a mixture of monomer component A) and filler component B), wherein the fissure sealant comprises
a) 25 to 80 wt.-% of at least one at least difunctional urethane (meth)acrylate, in particular 30 to 75 wt.-%,
b) 8 to 20 wt.-% of at least one hydrophilic alkylene oxide-based difunctional (meth)acrylate-based crosslinker comprising i) no urethane (meth)acrylate and/or ii) no aromatic residues, or a mixture of at least two of these crosslinkers,
c) 3 to 20 wt.-% of acidic monomers, in particular a mixture thereof, comprising i) at least one olefinic carboxylic acid and/or at least one olefinic carboxylic anhydride in combination with
ii) at least one olefinic acid ester of a phosphoric acid and/or at least one olefinic acid ester of a thiophosphoric acid, in particular at least one monoester of phosphoric acid of the formula I and/or II, or a mixture of i) and ii),
d) 0.8 to 3 wt.-% of at least one initiator and/or initiator system, each having an absorption maximum of 420 to 550 nm and comprising a diketone and optionally at least one co-initiator selected from a tert-amine and a 1,2-methylenedioxybenzene, and optionally
B) 0 to 40 wt.-% of filler component comprising at least one dental glass or a mixture of dental glasses, in particular a dental glass content of up to 40 wt.-%, wherein the total composition of the fissure sealant is 100 wt.-%.

The weight ratio of acidic monomers i) to ii) is preferably from 1:1 to 2:1, preferably from 5:4 to 5:2, particularly preferably 5:4 to 5:3.

The weight ratio of dental glasses with particle sizes from 0.8 to 2.5 micrometers and particle sizes from 0.1 to 0.8 micrometers is preferably from 4:1 to 1:1, preferably from 3:1 to 1.5:1.

In combinations with low filler content or without filler components, the content of acidic monomers may preferably comprise c) 8 to 20 wt.-%, of a mixture of acidic monomers comprising i) at least one olefinic carboxylic acid and/or at least one olefinic carboxylic anhydride, in particular 1.5 to 8 wt.-%, in combination with ii) at least one olefinic acid ester of a phosphoric acid and/or at least one olefinic acid ester of a thiophosphoric acid, in particular 6.5 to 12 wt.-%.

Further preferred is a fissure sealant comprising monomer component A) or a mixture of monomer component A) and filler component B), wherein the fissure sealant comprises
a) 30 to 80 wt.-% of at least one at least difunctional urethane (meth)acrylate,
b) 8 to 16 wt.-% of at least one hydrophilic alkylene oxide-based difunctional (meth)acrylate-based crosslinker which i) does not comprise urethane (meth)acrylate and/or ii) does not comprise aromatic residues, or a mixture of at least two of these crosslinkers,
c) 3 to 15 wt.-% of acidic monomers, in particular a mixture of i) and ii) comprising
   i) at least one olefinic carboxylic acid and/or at least one olefinic carboxylic anhydride, and
   ii) at least one olefinic acid ester of a phosphoric acid and/or at least one olefinic acid ester of a thiophosphoric acid, in particular at least one monoester of phosphoric acid of the formula I and/or II, or a mixture of i) and ii),
d) 0.8 to 3 wt.-% of at least one initiator system each having an absorption maximum of 420 to 550 nm and comprising a diketone and at least one co-initiator selected from a tert-amine and a 1,2-methylenedioxybenzene, and optionally
B) 0 to 40 wt.-% of a filler component comprising at least one dental glass or a mixture of dental glasses,
wherein the total composition of the fissure sealant is 100 wt.-%.

Particularly preferred b) hydrophilic alkylene oxide-based difunctional (meth)acrylate-based crosslinkers include polyethylene glycol di(meth)acrylates, more preferred TEGDMA, and polypropylene glycol di(meth)acrylates with n=2 to n=20, most preferred n=2 to 15, n=2 to 10, most preferred 2 to 8 or 5 to 9, linear or branched propylene glycol groups, preferred are tripropylene glycol dimethacrylate, tripropylene glycol diacrylate, tetrapropylene glycol dimethacrylate, tetrapropylene glycol diacrylate, pentapropylene glycol dimeth acrylate, pentapropylene glycol diacrylate, hexapropylene glycol dimethacrylate, hexapropylene glycol di acrylate, hepta (propylene glycol) dimethacrylate, hepta (propylene glycol) diacrylate, octapropylene glycol dimethacrylate, octapropylene glycol diacrylate, nonapropylene glycol dimethacrylate, nonapropylene glycol diacrylate, decapropylene glycol dimethacrylate, decapropylene glycol diacrylate, undeca propylene glycol dimethacrylate, undecapropylene glycol diacrylate, dodecapropylene glycol dimethacrylate, dodecapropylene glycol diacrylate, and mixtures of at least two of the foregoing di (meth)acrylates, and/or 1,3-butylene glycol dimethacrylate (butanediol di methacrylate), 1,3-butanediol dimethacrylate, hexanediol dimethacrylate, hexanediol glycol diacrylate, octanediol dimethacrylate, octadiol diacrylate, decanediol dimethacrylate, decanediol diacrylate, dodecanediol dimethacrylate, dodecanediol diacrylate and/or mixtures of at least two of the crosslinkers.

Particularly preferred are fissure sealers comprising polypropylene glycol di(meth)acrylates with n=2 to n=20, in particular n=2 to 15, n=5 to 12, preferably with n=2 to 9, particularly preferably with n=2 to 9, are used as b) hydrophilic alkylene oxide-based difunctional (meth)acrylate-based crosslinkers, such as PPGDA (polypropylene glycol diacrylate) with n=2 to 9, PEGDMA (polyethylene glycol dimethacrylate) with n=2 to 9, PPGDMA (polypropylene glycol dimethacrylate) with n=2 to 9 or DDDMA (dodecanediol dimethacrylate), and iii) BDMA, 1,4-butanediol dimethacrylate (1,4-BDMA) or pentaerythritol tetraacrylate.

In monomer component A), the a) at least one at least difunctional urethane (meth)acrylate is preferably selected from difunctional urethane (meth)acrylate with a divalent alkylene group comprising difunctional urethane (meth)acrylates with a linear or branched divalent alkylene group having 3 to 20 C atoms, urethane dimethacrylate functionalized ethers with a linear or branched divalent alkylene group having 3 to 20 C atoms, urethane dimethacrylate functionalized polyethers with linear or branched bivalent alkylene group with 3 to 20 C atoms, urethane di-acrylate oligomer, bis (methacryloxy-2-ethoxycarbonylamino) alkylene and/or bis (meth-acryloxy-2-ethoxycarbonylamino) substituted alkylene ethers are preferred, 1,6-bis (methacryloxy-2-ethoxycarbonylamino)-2,4,4-trimethylhexane (UDMA) is particularly preferred.

The difunctional urethane (meth)acrylate with bivalent alkylene group is preferably selected from linear or branched urethane dimethacrylates functionalized with a bivalent alkylene group, urethane dimethacrylate functionalized ethers or polyethers having alkylene group(s), such as bis (methacryloxy-2-ethoxycarbonylamino) alkylene, bis (meth-acryloxy-2-ethoxycarbonyl-amino) substituted polyalkylene ethers, preferably 1,6-bis (methacryloxy-2-ethoxycarbonyl-amino)-2,4,4-trimethylhexane. Preferred is a bis (methacryloxy-2-ethoxycarbonylamino) alkylene, wherein alkylene comprises linear or branched C3 to C20, preferably C3 to C6, as particularly preferred an alkylene substituted with methyl groups, such as HEMA-TMDI. The divalent alkylene preferably comprises 2,2,4-trimethylhexamethylene and/or 2,4,4-trimethylhexamethylene. Particularly preferred is UDMA (1,6-bis (methacryloxy-2-ethoxy-carbonylamino)-2,4,4-trimethylhexane), in particular HEMA-TDMI.

Also an object of the invention is a fissure sealant, preferably comprising:
b) at least two hydrophilic alkylene oxide-based difunctional (meth)acrylate-based crosslinkers, each selected from the combinations i) and ii) or i) and iii) or i), ii) and iii), particularly preferred is b) at least one mono-, di-, tri-, tetra-, poly-functional alkylene oxide-based difunctional (meth)acrylate-based crosslinker,
i) 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, 1,2-ethanediol diacrylate, diethylene glycol diacrylate, triethylene glycol diacrylate, tetraethylene glycol diacrylate, propylene glycol diacrylate, dipropylene glycol dimethacrylate, tripropylene glycol dimethacrylate, tetrapropylene glycol dimethacrylate dipropylene glycol diacrylate, tripropylene glycol diacrylate, tetrapropylene glycol diacrylate, and/or
ii) polyethylene glycol di(meth)acrylate and/or polypropylene glycol di(meth)acrylate or mixtures thereof, in particular with at least five ethylene glycol or propylene glycol groups, in particular polyethylene glycol di(meth)acrylates with n=5 to 20, in particular 5 to 15, ethylene glycol groups, and polypropylene glycol di(meth)acrylates with n=5 to n=20, in particular n=5 to 15, ethylene glycol groups, n=5 to 12, in particular 5 to 9, ethylene glycol groups, and polypropylene glycol di(meth)acrylates with n=5 to n=20, in particular n=5 to 15, n=5 to 12, in particular 5 to 9, linear or branched propylene glycol groups, pentapropylene glycol dimethacrylate is preferred, pentapropylene glycol diacrylate, hexapropylene glycol dimethacrylate, hexapropylene glycol diacrylate, hepta (propylene glycol) dimethacrylate, hepta (propylene glycol) diacrylate, octapropylene glycol dimethacrylate, octapropylene glycol diacrylate, nonapropylene glycol dimethacrylate, nonapropylene glycol diacrylate, decapropylene glycol dimethacrylate, decapropylene glycol diacrylate, undecapropylene glycol dimethacrylate, undecapropylene glycol diacrylate, dodecapropylene glycol dimethacrylate, dodecapropylene glycol diacrylate, and mixtures of at least two of the above-mentioned di (meth)acrylates, and/or
(iii) 1,3-butylene glycol dimethacrylate (butanediol dimethacrylate), hexanediol dimethacrylate, octanediol dimethacrylate, decanediol dimethacrylate, dodecanediol dimethacrylate.

Likewise, the fissure sealant may comprise: (b) at least one tri-, tetra-, penta-, and/or hexa-functional (meth)acrylate-based crosslinker other than urethane (meth)acrylate and selected from (i) having three (meth)acrylate groups of trimethylolpropane trimethacrylate, ethoxylated-(15)-trimethylolpropane triacrylate, ethoxylated-5-pentaerythritol triacrylate, propoxylated-(5.5)-glyceryl triacrylate, trimethylolpropane trimethacrylate, and/or (ii) with four (meth)acrylate groups from di-trimethylolpropane tetra-acrylate, ethoxylated-(4)-pentaerythritol tetra-acrylate, pentaerythritol tetra-acrylate, di-trimethylol propane tetramethacrylate, ethoxylated-(4)-pentaerythritol tetra-acrylate, pentaerythritol tetra-methacrylate and/or (iii) with five (meth)acrylate groups from di-pentaerythritol pentaacrylate, i-pentaerythritol pentamethacrylate, dipentaerythritol pentaacrylate, di (tetramethylol methane) pentameth-acrylate and/or (iv) with six (meth)acrylate groups a dipentaerythritol hexa (meth)acrylate.

The c) mixture of acidic monomers preferably comprises i) at least one olefinic carboxylic acid and/or at least one olefinic carboxylic acid anhydride comprising, in particular selected from, maleic acid, p-vinylbenzoic acid, 11-(meth)acryloyloxy-1,1-undecanedicarboxylic acid (MAC-10), 1,4-di (meth)acryloyloxyethylpyromellitic acid, 6-(meth)acryloyloxyethylnaphthalene-1,2,6-tricarboxylic acid, 4-(meth)acryloyloxymethyl-trimellitic acid and anhydrides thereof, 4-(Meth) acryloyloxyethyltrimellitic acid and anhydride thereof, 4-(Meth) acryloyloxybutyltrimellitic acid and anhydride thereof, 4-[2-hydroxy-3-(meth)acryloyloxy]butyltrimellitic acid and anhydride thereof, 2,3-bis (3,4-dicarboxybenzoyl oxy) propyl (meth)acrylate, 2—, 3-, or 4-(meth)acryloyloxybenzoic acid, N—O-di (meth)acryloyl oxytyrosine, O-(meth)acryloyloxy-tyrosine, N-(meth)acryloyloxytyrosine, N-(meth)acryloyloxy phenylalanine, N-(meth)acryloyl-p-aminobenzoic acid, N-(meth)acryloyl-O-aminobenzoic acid, adduct of glycidyl (meth)acrylate with N-phenylglycine or N-tolylglycine, 4-[(2-hydroxy-3-(meth)acryloyloxy-propyl) amino]-phthalic acid, 3- or 4-[N-methyl-N-(2-hydroxy-3-(meth)acryloyl oxypropyl) amino]phthalic acid, (meth)acryloylaminosalicylic acid and (meth)acryloyloxy salicylic acid, olefinic carboxylic acid having at least two carboxyl groups and/or olefinic carboxylic anhydrides having at least three carboxyl groups, in particular selected from dicarboxylic acids, tricarboxylic acids, tetracarboxylic acids, addition product of 2-hydroxyethyl (meth)acrylate and pyromellitic dianhydride (PMDM), an addition reaction product of 2 moles of hydroxyethyl (meth)acrylate and 1 mole of maleic anhydride or 3,3′,4,4′-benzophenonetetracarbon acid dianhydride (BTDA) or 3,3′,4,4′-biphenyltetracarbon acid dianhydride, and 2-(3,4-dicarboxybenzoyl-oxy) 1,3-di (meth)acryloyloxypropane,
in combination with ii) at least one olefinic acid monoester of a phosphoric acid and/or at least one olefinic acid monoester of a thiophosphoric acid, in particular at least one phosphoric acid-based (meth)acrylate, monoester of a phosphoric acid and at least one urethane (meth)acrylate, in particular monoester of phosphoric acid of formula I and/or II, and/or at least one thiophosphoric acid-based (meth)acrylate comprising 2-(meth)acryloyloxyethyl acid phosphate, 2- and 3-(meth)acryloyloxypropyl acid phosphate, 4-(meth)acryloyloxybutyl acid phosphate, 6-(meth)acryloyloxyhexyl acid phosphate, 8-(meth)acryloyloxyoctyl acidephosphate, 10-(meth)-acryloyloxydecylic acid phosphate, 12-(meth)acryloyloxydodecylic acid phosphate, bis (2-(meth)acryloyloxyethyl acid phosphate, bis-2 or 3-(meth)acryloyloxy propylic acid phosphate, 2-(meth)acryloyloxyethylphenylic acid phosphate, 2-(meth)-acryloyloxy ethyl-p-methoxyphenyl acid phosphate, and the corresponding thiophosphonates of the aforementioned (meth)acrylates.

A particularly preferred fissure sealant contains c) acidic monomers, in particular a mixture of acidic monomers comprising, in particular selected from i) 4-methacryloyloxyethyltrimellitic acid (4-MET) and/or its anhydride (4-META), and
ii) acidic monoester(s) of at least one phosphoric acid comprising at least two urethane (meth)acrylate groups, in particular wherein at least two urethane (meth)acrylate groups are covalently bonded to an O atom of the phosphoric acid by means of a group having at least one trivalent C atom and optionally O atoms, in particular. OH₂C—C(—O)—CHO₂: group, and form the monoester of the phosphoric acid, wherein the urethane (meth)acrylate groups are independently selected from urethane methacrylate and urethane acrylate groups, 2-methacryloyloxyethylphenylic acid phosphate, 10-methacryloyloxydecylic acid phosphate, 2-acryloyloxyethylphenylic acid phosphate and/or 10-acryloyloxydecylic acid phosphate, and mixtures comprising at least two of the aforementioned monoesters.

In one alternative, at least two urethane (meth)acrylate groups are covalently bonded via one O atom each to a group comprising a quaternary C atom and at least one monoester of a phosphoric acid. The group comprising a trivalent or quaternary C atom preferably comprises 3 to 6 C atoms, and in particular the respective urethane (meth)acrylate group and the monoester of phosphoric acid can each be covalently bonded to the group independently via a polyalkylene oxide group. polyalkylene oxide comprises polyethylene oxide, polypropylene oxide, polyalkylene oxide groups comprising propylene oxide and ethylene oxide groups, blocks of polypropylene oxide and polyethylene oxide.

Quite particularly preferred ii) acidic monoesters of at least one phosphoric acid comprise at least one phosphoric acid of the general formulas I, Ia, Ib and/or II or mixtures of at least two of these monoesters

### R²

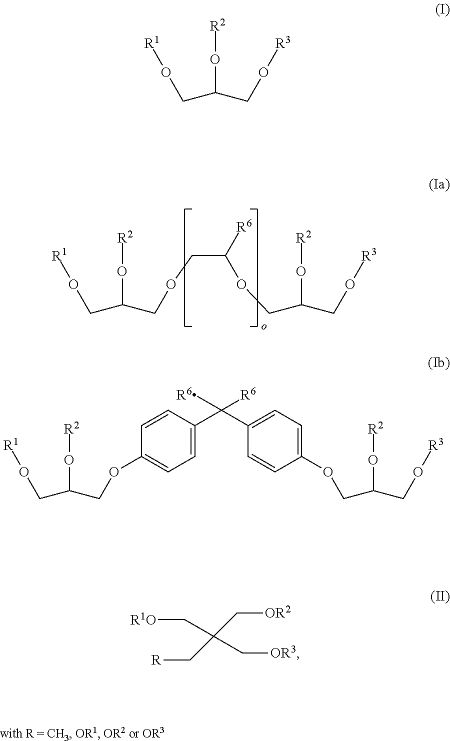

wherein R¹, R² and R³ are selected from the formulas III, IIIa, IIIb, IV, V, VI=VIa and/or VIb and VII=VIIa and/or VIIb, VIII=Villa and/or VIIIb with

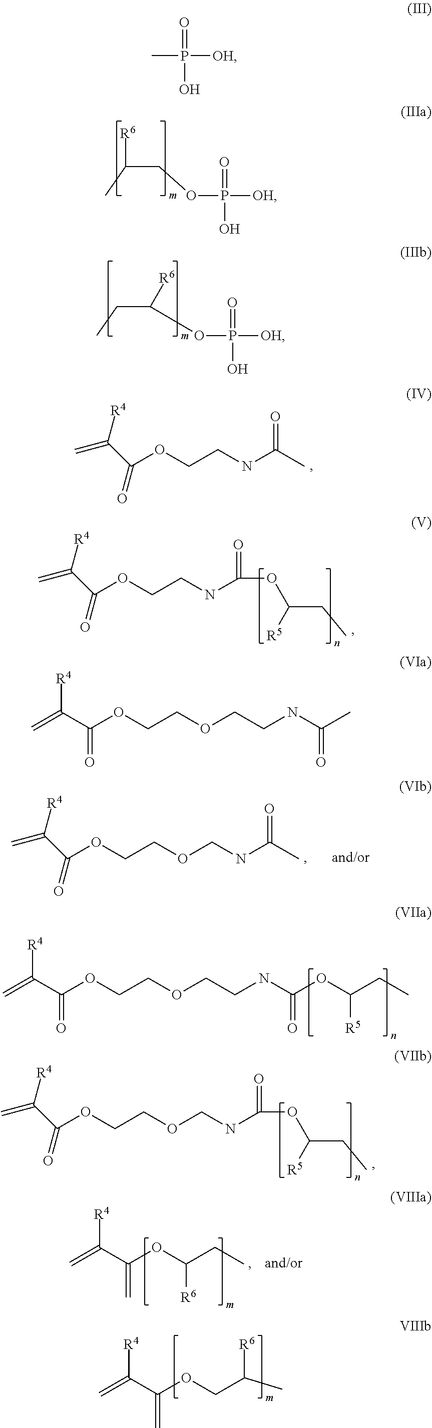

where, in formulas V and VII, n is in each case independently 1 to 100, in particular n=1 to 20, and wherein at least one or two residues selected from R¹, R² and R³ correspond to a group of the formula III, IIIa and/or IIIb and the remaining residue or residues of R¹, R² and R³ of formulas I and/or II are selected from a group of formula IV, V, VIa, VIb, VIIa, VIIb, VIIIa and/or VIIIb, wherein R⁴ and R⁵ are each independently selected from H, methyl and ethyl.

In an alternative, the remaining groups may be selected from at least two formulas of IV, V, VIa and/or VIIa. Preferably, in formula I and/or II, one residue corresponds to the residues R¹, R² and R³ of formula III and the remaining residues each correspond to a group of formula IV, V, VIa or VIIa, preferably the remaining residues selected from R¹, R² and R³ correspond to identical groups of formulas IV, V, VIa or VIIa. Further preferably R² corresponds to formula III and the remaining are identical groups selected from formulas IV, V, VIa, VIIa, preferably IV. A preferred monoester corresponds to formula I with R² equal to formula III and R¹ and R² equal to formula IV or VIIa.

Particularly preferred in the above formulas is I to VIIIb:
wherein o=1 to 100 in formula Ia, in particular o=1 to 5,
with m=1 to 100 in formulas IIIa and/or IIIb, in particular 1 to 20, wherein in formula IIIa and IIIb R⁶ is selected from H and methyl, and wherein the divalent alkylene oxide group comprising R⁶ may also be mirrored or laterally reversed,
wherein in the formulas V, VIIa and/or VIIb in each case independently n=1 to 100, in particular n=1 to 20, and
wherein in formulas I, Ia, Ib and II at least one residue or two residues selected from R¹, R² and R³ correspond to a group of the formula III, IIIb and/or IIIa and the residue(s) remaining residues of R¹, R² and R³ are selected from at least one group of the following formulas IV, V, VIa, VIb, VIIa, VIIb, VIIIa and/or VIIIb, wherein R⁴ and R⁵ are each independently in the groups of the formulas IV, V, VIa, VIb, VIIa, VIIb, VIIIa and VIIIb are selected from H, methyl and ethyl, in particular the remaining groups are selected from at least two formulas of IV, V, VIa, VIIb, VIIa, VIIb, VIIIa and/or VIIIb, and
wherein each R⁶ is independently selected from the groups of formulas Ia, Ib, IIIa, IIIb, VIIIa and VIIIb is selected from H and methyl,
wherein m=0 or 1 to 100 in formulas VIIIa and/or VIIIb, in particular 1 to 20, with R⁶ is selected from H and methyl.

Typical examples of monophosphoric acid, which may also be present as dual monophosphoric acids, are exemplified below without limiting the disclosure to these examples:

In the monoesters of a phosphoric acid shown below, R^{10A} and R^{10B}/R^{10C} are selected from H and methyl, preferably R^{10A}, R^{10B} and R^{10C} are each H or methyl. In particular with n=0 or 1 to 100, preferably n=0 or 1 to 10.

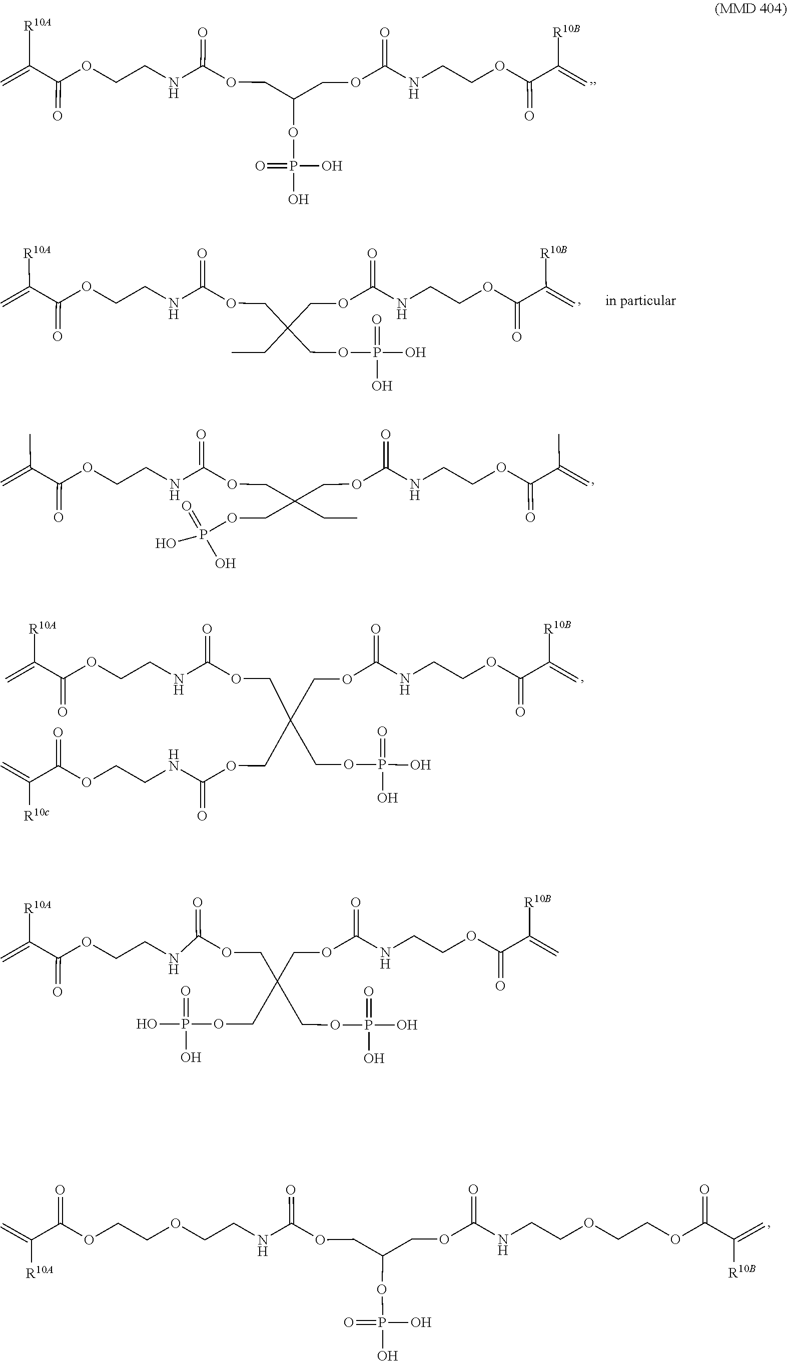

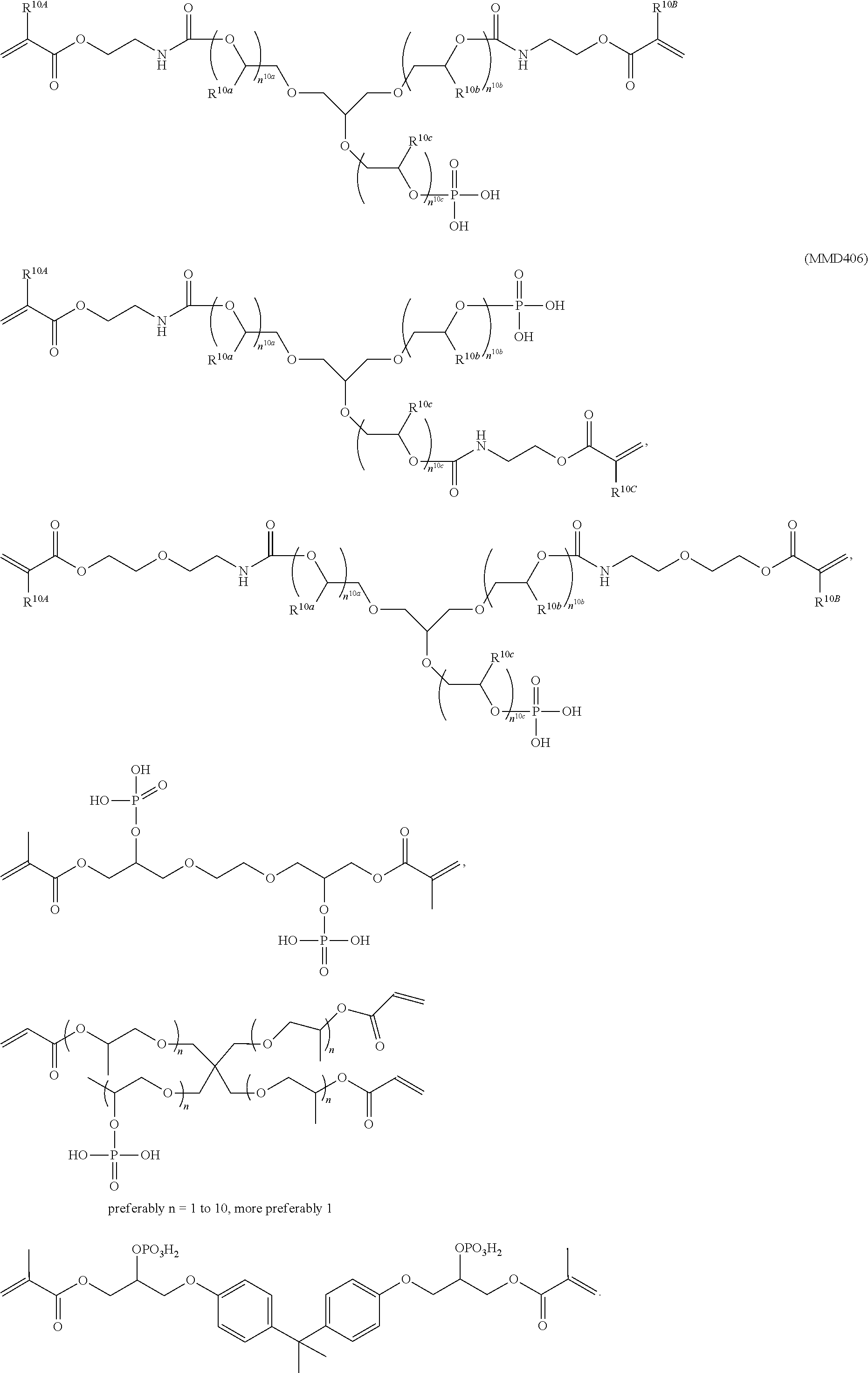

Particularly preferred are 11-(meth)acryloyloxy-1,1-undecanedicarboxylic acid (MAC-10) and 4-methacryloyloxyethyltrimellitic acid (4-MET) and/or its anhydride (4-META), and at least one monoester of a phosphoric acid and at least one urethane (meth)acrylate, particularly preferred are the monoesters of the phosphoric acids of the formulas I and II, 2-(meth)acryloyl oxyethylphenylic acid phosphate and/or 10-(meth)acryloyloxydecyl acid phosphate.

The content of the acidic monomers MDP and 4-META and/or 4-MET can be very low in the monomer component and yet very good adhesion values are achieved with the fissure sealant according to the invention. Thus, the content of MDP can be approx. 4 to 5 wt.-% and the content of 4-META and/or 4-MET approx. 2 wt.-% in the monomer component, whereby preferably an adhesion to dentin (bovine tooth) in the range of 16 to 21 MPa and to enamel (bovine tooth) in the range of 4 to 21 MPa can be measured (DIN: ISO 29022).

Furthermore, in preferred embodiments it may be important to realize a high opacity of the bright white colored fissure sealant, since such a material is only used in a thin layer. This is because if such a demarcation material were to exhibit the same opacity as a conventional flow composite, it would not be recognizable or only insufficiently recognizable on the tooth structure despite its white color. Setting a correspondingly high opacity is technically not a problem in principle, but the subsequent light-curing of the material becomes problematic with increasing opacity. The more opaque a light-curing material is, the smaller the layer thicknesses that can be polymerized in one polymerization cycle.

In further alternatives, the fissure sealant in the A) monomer component may comprise at least one monofunctional (meth)acrylate-based monomer, excluding 2-hydroxyethylmethyl acrylate (HEMA). Alternatively or additionally suitable are also urethane bond-containing methacrylates, such as 2-(meth)acryloyloxy-ethyl isocyanate. Other suitable monofunctional (meth)acrylate-based monomers include aromatic vinyl compounds, such as styrene and divinylbenzene, vinyl esters, such as vinyl acetate, aliphatic esters of (meth)acrylic acid, such as methyl (meth)acrylate, ethyl (meth)acrylate, propyl (meth)acrylate, butyl (meth)acrylate, pentyl (meth)acrylate, hexyl (meth)acrylate.

As B) filler component, the fissure sealant preferably comprises at least one a) dental glass or a mixture of dental glasses, and b) optionally comprising at least one inorganic fluoride and/or amorphous metal oxide, mixed oxide, crystalline metal oxide, silicate, wherein a) the at least one dental glass comprises aluminosilicate glasses or fluoroaluminosilicate glasses, barium aluminum silicate, barium aluminum boron fluorosilicate, strontium silicate, strontium borosilicate, lithium silicate and/or lithium aluminum silicate, dental glasses containing ytterbium fluoride, and mixtures of at least two of the aforementioned dental glasses, and b) comprises ytterbium fluoride, amorphous spherical fillers based on oxide or mixed oxide, such as amorphous SiO₂, ZrO₂ or also mixed oxides of SiO₂ and ZrO₂, quartz, feldspar, fumed or precipitated silicas, amorphous silicas, phyllosilicates, and mixtures of at least two of the fillers. The fillers may all preferably be silanized. Common silanizing agents include (meth)acryloyloxypropyltrimethoxysilane and (meth)acryloyloxypropyltriethoxysilane or (meth)acryloyloxymethyltrimethoxysilane and (meth)acryloyloxymethyltriethoxysilane.

According to a preferred embodiment, the dental fissure sealant comprises at least one dental glass and/or mixtures thereof, in particular a radiopaque dental glass or mixtures thereof, of an average particle size d₅₀ of 0.2 to 5.0 μm, preferably with d₅₀ of 0.2 to 2.0 μm, preferably with an average particle size of 0.2 to 1.75 μm, in particular with d₅₀ of 1.0 μm optionally plus/minus 0.6 μm. A particularly preferred dental glass comprises barium aluminum borosilicate glass, more preferred barium aluminum bor fluorosilicate. Furthermore, a barium aluminum silicate glass with a refractive index of n=1.52 to 1.55, preferably 1.53.

Furthermore, it is an object of the invention to provide a dental fissure sealant comprising a filler component comprising at least one dental glass or a mixture of dental glasses comprising barium aluminum borosilicate glass, barium aluminum borofluorosilicate glass, in particular silanized, preferably functionalized with methacryloxypropyl groups, and optionally at least one non-agglomerated amorphous metal oxide of a primary particle size of 2 to 45 nm, the amorphous metal oxide comprising precipitated silica, zirconia, mixed oxides or mixtures thereof, in particular the metal oxides being silanized.

As initiator or initiator component, preferably d) the diketone is a 1,7,7-trimethyl-bicyclo-[2.2.1]-hepta-2,3-dione (champherquinone), phenylpropanedione, benzoyltrimethyl german (BTMGe) and/or dibenzoyldiethylgerman (DBDEGe), and the co-initiator preferably comprises 2-n-butoxyethyl-4-(dimethylamino) benzoate and/or as 1,2-methylenedioxybenzene piperonyl alcohol (1,2-methylenedioxybenzene-4-methanol). Other suitable tert-amines may include 2-(ethylhexyl)-4-(N,N-dimethylamino) benzoate, dimethylaminobenzoic acid ester, triethanol-amines, N,N-3,5-,N,N-3,5-tetramethyl aniline, 4-(dimethylamino) phenylethyl alcohol, dimethylamino benzoic acid ester, 4-(N,N-dimethylamino) benzoic acid.

In a particularly preferred embodiment, the fissure sealant contains from 0.2 to 2.5 wt.-%, preferably from 0.25 wt.-% to 2.5 wt.-%, particularly preferably from 0.3 to 2.5 wt.-% of at least one diketone selected from 1,7,7-trimethyl-bicyclo-[2.2.1]-hepta-2,3-dione (champher-quinone), phenylpropanedione, benzoyltrimethylgerman (BTMGe) and/or dibenzoyldiethyl-german (DBDEGe), and from 0.5% to 2.5 wt.-%, preferably from 0.55% to 2.5 wt.-%, particularly preferred from 0.6 to 2.5 wt.-% of at least one co-initiator selected from tert-amine and 1,2-methylenedioxybenzenes, in particular selected from 2-n-butoxyethyl-4-(dimethylamino) benzoate and/or piperonyl alcohol, wherein the mass ratio of diketone to co-initiator selected from tert-amine or 1,2-methylenedioxybenzenes is from 1:1.5 to 1:3, preferably from 1:1.75 to 1:3. Particularly preferred are from 0.25 to 2.5 wt.-% diketone and from 0.55 to 2.5 wt.-% co-initiator, with diketone to co-initiator from 1:1.75 to 1:2.25.

At least one further pigment, a dye, and in particular at least one UV and/or Vis stabilizer comprising 2-hydroxy-4-methoxybenzophenone may be present both in the monomer component and/or in the filler component. The pigment is added at its content to the filler component.

The c) acidic monomers, in particular the mixture of acidic monomers, preferably comprises i) at least one olefinic carboxylic acid and/or the at least one olefinic carboxylic anhydride, preferably at least one carboxylic acid functionalized and/or carboxylic anhydride functionalized (meth)acrylate monomer, and ii) at least one olefinic acidic ester of phosphoric acid, in particular a monoester of a phosphoric acid, and/or at least one olefinic acid ester of a thiophosphoric acid, in particular a monoester of a thiophosphoric acid, preferably at least one monoester of a phosphoric acid and of a (meth)acrylate, monoester of a phosphoric acid and of a urethane (meth)acrylate, in particular at least one monoester of phosphoric acid of the formula I and/or II, and/or at least one monoester of a thiophosphoric acid and of a (meth)acrylate, which are present in a mass ratio of 1:3 to 1:1, preferably from 1:2.75 to 1:1. 75, preferably from 1:2.25 to 1:1.75. The above definition that (meth)acrylate comprises both the acrylate or the methacrylate also applies herein.

Preferably, a fissure sealant c) comprises a mixture of acidic monomers comprising i) at least one olefinic carboxylic acid and/or at least one olefinic carboxylic anhydride, in combination with ii) at least one olefinic acidic ester of a phosphoric acid, in particular of the formula I and/or II, and/or at least one olefinic acidic ester of a thiophosphoric acid, i) and ii) being present in a weight ratio of 1:1 to 2:1, in particular 7:6 to 5:2, preferably from 5:4 to 5:2.

In a particularly preferred embodiment, the fissure sealant comprises in the A) monomer component in c) as i) 2 to 10 wt.-%, in particular 3.75 to 10 wt.-%, of at least one olefinic carboxylic acid and/or the at least one olefinic carboxylic anhydride, preferably at least one carboxylic acid-functionalized and/or carboxylic anhydride-functionalized (meth)acrylate monomer, and as ii) 4 to 20 wt.-%, in particular 9.5 to 20% of at least one olefinic acid ester of a phosphoric acid and/or at least one olefinic acid ester of a thiophosphoric acid, preferably at least one monoester of a phosphoric acid and of a (meth)acrylate, monoester of a phosphoric acid and of at least one urethane (meth)acrylate, in particular monoester of phosphoric acid of the formula I and/or II, and/or at least one monoester of a thiophosphoric acid and of a (meth)acrylate, with respect to the total composition of 100 wt.-% of % of the A) monomer component.

According to a particularly preferred embodiment, a fissure sealant A) optionally contains the monomer component in admixture with B) the filler component, wherein the fissure sealant comprises
a) 30 to 80 wt.-% of at least one at least difunctional urethane (meth)acrylate,
b) 8 to 20 wt.-% of at least one hydrophilic alkylene oxide-based di-functional (meth)acrylate-based crosslinker comprising i) no urethane (meth)acrylate and/or ii) no aromatic residues, or a mixture of at least two of these crosslinkers,
c) i) 2 to 10 wt.-% of at least one olefinic carboxylic acid and/or at least one olefinic carboxylic anhydride, in particular at least one carboxylic acid-functionalized and/or carboxylic anhydride-functionalized (meth)acrylate monomer, and
ii) from 4 to 20 wt.-% of at least one olefinic acid ester of a phosphoric acid and/or at least one olefinic acid ester of a thiophosphoric acid, in particular at least one monoester of a phosphoric acid and a (meth)acrylate, monoester of a phosphoric acid and a urethane (meth)acrylate and/or at least one monoester of a thiophosphoric acid and a (meth)acrylate, in particular at least one monoester of phosphoric acid of the formula I and/or II, or a mixture of i) and ii),
d) 0.3 to 2.5 wt.-% of at least one diketone selected from 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe) and 0.6 to 2.5 wt.-% of at least one co-initiator selected from tert-amine and 1,2-methylenedioxybenzenes, in particular selected from 2-n-butoxyethyl-4-(dimethylamino) benzoate and/or piperonyl alcohol, wherein the mass ratio of diketone to co-initiator selected from tert-amine or 1,2-methylenedioxybenzenes is from 1:1.5 to 1:3,
e) 0 to 1 wt.-% of at least one pigment comprising titanium dioxide, and 0 to 2 wt.-% of dye, UV and/or Vis stabilizer, and
f) 0 to 30 wt.-% of at least one dental glass, in particular a content of up to 30 wt.-%,
g) 0 to 10 wt.-% of inorganic fluoride, such as ytterbium fluoride, metal oxide, mixed oxides and/or silicates,
h) 0 to 1 wt.-%, in particular 0.1 to 0.5 wt.-% of at least one pigment comprising titanium dioxide, and 0 to 2 wt.-% of dye, UV and/or Vis stabilizer, and/or mixtures of at least two of the fillers e) to h), the above components a) to h) giving the total composition of 100 wt.-% of the fissure sealant.

Further, a fissure sealant is preferred which comprises as.
A) monomer component comprising
   a) 50 to 80 wt.-% of at least one at least difunctional urethane (meth)acrylate,
   b) 10 to 35 wt.-%, in particular 15 to 25 wt.-%, of at least one hydrophilic alkylene oxide-based difunctional (meth)acrylate-based crosslinker which i) does not comprise urethane (meth)acrylate and/or ii) does not comprise aromatic residues, or a mixture of at least two of these crosslinkers, and/or
10 to 35 wt.-%, in particular 15 to 25 wt.-%, of at least one tri-, tetra-, penta- and/or hexa-functional (meth)acrylate-based crosslinker which is not a urethane (meth)acrylate, or 10 to 35 wt.-% of a mixture of alkylene oxide-based difunctional (meth)acrylate-based crosslinkers and at least one tri-, tetra-, penta- and/or hexa-functional (meth)acrylate-based crosslinker,
c) i) 2 to 10 wt.-%, in particular 3.75 to 10 wt.-% of at least one olefinic carboxylic acid and/or olefinic carboxylic anhydride, in particular at least one carboxylic acid-functionalized and/or carboxylic anhydride-functionalized (meth)acrylate monomer, and
ii) 4 to 20 wt.-%, in particular 9.5 to 20 wt.-%, of at least one olefinic acid ester of a phosphoric acid, monoesters of a phosphoric acid and of at least one urethane (meth)acrylate and/or at least one olefinic acid ester of a thiophosphoric acid, in particular at least one monoester of a phosphoric acid, in particular monoesters of phosphoric acid of the formula I and/or II, and of a (meth)acrylate and/or at least one monoester of a thiophosphoric acid and of a (meth)acrylate, or a mixture of i) and ii),
d) 0.3 to 2.5 wt.-%, in particular 0.5 to 2.5 wt.-%, of at least one diketone selected from 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe) and 0.6 to 2.5 wt.-%, in particular 1.0 to 2.5 wt.-%, of at least one co-initiator selected from tert-amine and 1,2-methylenedioxybenzenes, in particular selected from 2-n-butoxyethyl-4-(dimethylamino) benzoate and/or piperonyl alcohol (1,2-methylenedioxybenzene-4-methanol), the mass ratio of diketone to co-initiator selected from tert-amine and 1,2-methylenedioxybenzenes, preferably a tert-amine, being 1:1.5 to 1:3, preferably 1:2, the value 2 being provided with a variation of +/−0.15,
e) 0 to 1 wt.-%, in particular 0.1 to 0.5 wt.-%, of at least one pigment comprising titanium dioxide, and optionally 0 to 2 wt.-% of dye, UV stabilizer and/or vis stabilizer, wherein the total composition of monomer component A) is 100 wt.-%, and optionally B) a filler component comprising
a) 90 to 100 wt.-% of at least one dental glass or a mixture of dental glasses,
b) 0 to 10 wt.-%, in particular 5 to 10 wt.-%, of inorganic fluoride, such as ytterbium fluoride, metal oxide, such as SiO₂, mixed oxides, silicates, quartz, feldspar and/or mixtures thereof,
c) 0 to 1 wt.-%, in particular 0.1 to 0.5 wt.-%, of at least one pigment comprising titanium dioxide, and optionally 0 to 2 wt.-% of dye, UV stabilizer and/or vis stabilizer, and/or

Mixtures of at least two of the fillers, wherein the total composition of the filler component is 100 wt.-%, and, wherein A) the monomer component and B) the filler component are present in the fissure sealant in a mass ratio of from 60:40 to 100:0, and wherein the total composition of the fissure sealant is 100 wt.-%.

Likewise, according to a particularly preferred embodiment, an object of the invention is a fissure sealant having a A) monomer component comprising.
a) 50 to 80 wt.-% of at least one at least difunctional urethane (meth)acrylate,
b) 10 to 35 wt.-%, in particular 15 to 25 wt.-%, of at least one hydrophilic alkylene oxide-based difunctional (meth)acrylate-based crosslinker which i) does not comprise urethane (meth)acrylate and/or ii) does not comprise aromatic residues, or of a mixture of at least two of these crosslinkers, preferably at least one di- to polyalkylene oxide-based difunctional (meth)acrylate-based crosslinker,
(c) (i) 2 to 10 wt.-%, in particular 3.75 to 10 wt.-%, of 4-(methacryloyl-oxyethyl) trimellitic anhydride (4-META) and/or 4-methacryloyloxyethyltrimellitic acid (4-MET), and
ii) from 4 to 20 wt.-%, in particular from 9.5 to 20 wt.-%, of monoesters of phosphoric acid of the formula I and/or II 2-(meth)acryloyloxyethylphenylic acid phosphate and/or 10-(meth)acryloyl oxydecyl acid phosphate, and/or a mixture of i) and ii),
d) 0.3 to 2.5 wt.-%, in particular 0.5 to 2.5 wt.-%, of at least one diketone selected from 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe) and 0.6 to 2.5 wt.-%, in particular 1.0 to 2.5 wt.-%, of at least one co-initiator selected from 2-n-butoxyethyl-4-(dimethylamino) benzoate and piperonyl alcohol (1,2-methylenedioxy benzen-4-methanol), in particular wherein the mass ratio of diketone to co-initiator is is 1:1.5 to 1:3, preferably 1:2 with a variation of the value 2 of +/−0.15,
e) 0 to 0.5 wt.-% of at least one pigment comprising titanium dioxide, and 0 to 2 wt.-% of dye, UV stabilizer and/or vis stabilizer, wherein the total composition of monomer component A) is 100 wt.-%, and optionally
B) a filler component comprising
a) 90 to 100 wt.-% of at least one dental glass,
b) 0 to 10 wt.-%, in particular 5 to 10 wt.-%, inorganic fluoride, such as ytterbium fluoride, metal oxide, such as SiO₂, mixed oxides, silicate,
c) 0 to 0.5 wt.-% of at least one pigment comprising titanium dioxide and 0 to 2 wt.-% of dye, UV stabilizer and/or Vis stabilizer, and/or
mixtures of at least two fillers, and, wherein the total composition of the filler components is 100 wt.-%, and wherein A) the monomer component and B) the filler component are present in the fissure sealant in a mass ratio of from 60:40 to 100:0, and wherein the total composition of the fissure sealant is 100 wt.-%.

Another particularly preferred fissure sealant comprises as A) a monomer component comprising.
a) 50 to 80 wt.-% of at least one at least difunctional urethane (meth)acrylate,
b)—at least one hydrophilic alkylene oxide-based difunctional (meth)acrylate-based crosslinker selected from i) and ii) or i) and iii) or i), ii) and iii), comprising
i) 5 to 15 wt.-% of 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol dimethacrylate, tetrapropylene glycol dimethacrylate dipropylene glycol diacrylate, tripropylene glycol diacrylate, tetrapropylene glycol diacrylate, and
ii) 5 to 15 wt.-% of polyethylene glycol di(meth)acrylate and/or polypropylene glycol di (meth)acrylate or with at least five ethylene glycol or propylene glycol groups, and mixtures thereof, and/or iii) 5 to 15 wt.-% of 1,3-butylene glycol dimethacrylate, hexanediol dimethacrylate, octanediol dimethacrylate, decanediol dimethacrylate, dodecanediol dimethacrylate, and/or
5 to 15 wt.-% of at least one tri-, tetra-, penta- and/or hexa-functional (meth)acrylate-based crosslinker which is not urethane (meth)acrylate, the total content of i) and ii) or i) and iii) or i), ii) and iii) in monomer component A) being from 10 to 35 wt.-%,
c) i) 2 to 10 wt.-%, in particular 3.75 to 10 wt.-% of 4-(methacryloyloxyethyl)-trimellitic anhydride (4-META) and/or 4-methacryloyloxyethyltrimellitic acid (4-MET) and
ii) 4 to 20 wt.-%, in particular 9.5 to 20 wt.-%, of monoesters of phosphoric acid corresponding to formula I and/or II, 2-(meth)acryloyloxyethylphenylic acid phosphate and/or 10-(meth)acryloyloxydecyl acid phosphate, and/or a mixture of i) and ii),
d) 0.3 to 2.5 wt.-%, in particular 0.5 to 2.5 wt.-%, of at least one diketone selected from 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe) and 0.6 to 2.5 wt.-%, in particular 1.0 to 2.5 wt.-%, of at least one co-initiator selected from 2-n-butoxyethyl-4-(dimethylamino) benzoate and/or piperonyl alcohol (1,2-methylene dioxybenzene-4-methanol), the mass ratio of diketone to co-initiator being 1:1.5 to 1:3, preferably 1:2 with a variation of the value 2 by +/−0.15,
e) 0 to 0.5 wt.-% of at least one pigment comprising titanium dioxide, and 0 to 2 wt.-% of dye, UV stabilizer and/or vis stabilizer, wherein the total composition of monomer component A) is 100 wt.-%, and optionally
B) a filler component comprising
a) 90 to 100 wt.-% of at least one dental glass or a mixture of dental glasses,
b) 0 to 10 wt.-%, in particular 5 to 10 wt.-% of inorganic fluoride, such as ytterbium fluoride, metal oxide, such as SiO₂, mixed oxides, crystalline metal oxide, silicate,
c) 0 to 0.5 wt.-% of at least one pigment comprising titanium dioxide, and 0 to 2 wt.-% of dye, UV and/or Vis stabilizer, and/or mixtures of at least two fillers, and wherein the total composition of the filler components is 100 wt.-%. %, and/or a mixture of at least two of the fillers, and wherein the total composition of the filler component is 100 wt.-%, and wherein A) the monomer component and B) the filler component are present in the fissure sealant in a mass ratio of 60:40 to 100:0, and wherein the total composition of the fissure sealant is 100 wt.-%.

Also particularly preferred is a fissure sealant, in particular comprising A) the monomer component optionally in admixture with B) the filler component, wherein the fissure sealant comprises
a) 30 to 80 wt.-% of at least one at least difunctional urethane (meth)acrylate,
b) 8 to 20 wt.-% of at least one hydrophilic alkylene oxide-based difunctional (meth)acrylate-based crosslinker comprising i) no urethane (meth)acrylate and/or ii) no aromatic residues, or a mixture of at least two of these crosslinkers,
c) i) 2 to 10 wt.-% of at least one olefinic carboxylic acid and/or at least one olefinic carboxylic anhydride, in particular at least one carboxylic acid-functionalized and/or carboxylic anhydride-functionalized (meth)acrylate monomer, and
ii) 4 to 20 wt.-% of at least one olefinic acid ester of a phosphoric acid and/or at least one olefinic acid ester of a thiophosphoric acid, in particular at least one monoester of a phosphoric acid and a (meth)acrylate, monoester of a phosphoric acid and a urethane (meth)acrylate and/or at least one monoester of a thiophosphoric acid and a (meth)acrylate, or a mixture of i) and ii),
d) 0.3 to 2.5 wt.-% of at least one diketone selected from 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe) and
0.6 to 2.5 wt.-% of at least one co-initiator selected from tert-amine and 1,2-methylenedioxybenzenes, in particular selected from 2-n-butoxyethyl-4-(dimethylamino) benzoate and/or piperonyl alcohol, wherein the mass ratio of diketone to co-initiator selected from tert-amine or 1,2-methylenedioxybenzenes is from 1:1.5 to 1:3,
e) 0 to 1 wt.-% of at least one pigment comprising titanium dioxide, and 0 to 2 wt.-% of dye, UV stabilizer and/or Vis stabilizer, and
f) 0 to 30 wt.-% of at least one dental glass, in particular 1 to 30 wt.-%, preferably 10 to 30 wt.-%,
g) 0 to 10 wt.-% of inorganic fluoride, such as ytterbium fluoride, in particular ytterbium (III) fluoride, metal oxide, mixed oxides and/or silicates,
h) 0 to 1 wt.-%, in particular 0.1 to 0.5 wt.-%, of at least one pigment comprising titanium dioxide, and 0 to 2 wt.-% of dye, UV and/or Vis stabilizer, and/or mixtures of at least two of the fillers e) to h), the above components a) to h) giving the total composition of 100 wt.-% of the fissure sealant.

In the stated compositions in which the contents of the components are named, the contents named after in particular are to be understood as belonging together; the same applies in subgroups to the contents named there as preferred. The disclosure is also to be understood in connection with the examples not exhaustively listed and is addressed to the chemist or material scientist.

Also an object of the invention is a polymerized dental fissure sealant obtainable by polymerization, in particular by visible light polymerization, of a fissure sealant, in particular the polymerized fissure sealant has a shear bond strength to untreated bovine tooth enamel, and ground bovine tooth enamel of greater than or equal to 11 MPa, in particular greater than or equal to 13 MPa, preferably greater than or equal to 15 MPa, particularly preferably greater than or equal to 17 MPa, further preferably greater than or equal to 19 MPa, preferably greater than or equal to 22 MPa, particularly preferably greater than or equal to 23 MPa. It is further preferred if the fissure sealant comprising A) 80 to 100 wt.-% monomer component and B) 0 to 20 wt.-% filler component with a total composition of 100 wt.-% has a shear bond strength of greater than or equal to 15 MPa, preferably greater than or equal to 17 MPa, particularly preferably greater than or equal to 18 MPa, in particular on unground bovine tooth enamel after thermocycling and preferably without a separate etching step with a composition differing from that of the fissure sealant having been applied to the unground enamel prior to application of the fissure sealant according to the invention, i.e. on non-pre-etched bovine tooth enamel.

It is also an object of the invention to use a curable or polymerizable dental fissure sealant for radiation-curing, self-adhesive bonding to dentin and/or enamel of teeth, in particular human or veterinary teeth. Radiation curing is preferably carried out by means of visible light (Vis radiation), preferably in the wavelength range from 420 to 500 nm, preferably at 485 nm. Suitable light sources are LED illuminants.

Also an object of the invention is a self-adhesive, radiation-curable, dental fissure sealant or self-adhesive, polymerized, in particular radiation-cured, fissure sealant for use in sealing fissures in enamel and/or dentin in very fine fissures and/or horn, such as hooves, or for use for radiation-curing, self-adhesive adhesion to substrates comprising inorganic glass, PMMA, ceramics, hybrid ceramics, metals and/or alloys, etc, to dentin and/or enamel of teeth, in particular human or veterinary teeth and/or as an underfilling material.

The following examples are intended to explain the subject matter of the invention without limiting it to the specific examples.

### Method Description:

Shear bond strength was measured according to DIN EN: ISO 29022 (2013). Bovine tooth enamel and/or dentin surfaces were prepared on SiC paper of grit size 120 to grit size 320. In the case of “uncut” surfaces, bovine tooth enamel surfaces mechanically cleaned are used as the bonding surface (Sof-Lex discs from 3M). The fissure sealant is massaged onto the tooth substrate and exposed to a Translux Wave (Kulzer GmbH, spectrum: 440 to 480 nm, power density 1200 milliwatts/cm²) for 10 s. The material is then filled into cylindrical plastic molds (Ultradent equipment) and cured for 20 s. To simulate aging, some samples are subjected to thermal cycling (TWC=thermocyclic, thermal cycling bath) (5° C. to 55° C. water bath, 30 s dwell time and 5 s transfer time, 5,000 cycles). Shear bond strength is determined with a universal tester (crosshead speed 1 mm/min). 24 h measurements 37° C. in H₂O. Measurement universal tester (room temperature). Unless otherwise stated, BTE (bovine tooth enamel)/RTD (bovien tooth dentine) are the 24 h measurements. BTD (bovine tooth dentine).

Flexural strength and flexural modulus of elasticity are performed according to DIN-ISO standard 4049 (Polymer-based filling, restorative and luting materials, measurements at room temperature, 2019).

The determination of water absorption/solubility is carried out according to DIN-ISO 4049 section 7.12 to 4 to 8 test specimens with 15 mm diameter and 1 mm width are cured, dried, stored for 7 days at 37° C. in H₂O and dried again.

The film thickness of the fissure sealant is determined according to DIN-ISO 4049 7.5. The material is loaded between 2 glass plates with 150 N for 180 s, cured and the difference measured with an outside micrometer.

The hardness is determined as follows: Cure test specimen 10 mm Delrinform/Teflon mold for 20 s on the upper side (OS). Mark bottom side (US). Store for 24 h at 37° C. in H₂O, grind flat and determine the penetration force (N/mm²)/penetration depth of the top side/bottom side with Zwick Universal Hardness using diamond.

The alkylene oxide-based di-functional methacrylates used in the following examples, such as TEGDMA, PEGDMA or DDDMA (dodecanediol dimethacrylate), particularly in preferred alternatives due to the combination of these and/or due to the content in the unpolymerized state of the fissure sealant, cause good flow to the enamel as well as penetration into fissures and thus the high bonding performance after radiation curing. Adhesion to the enamel is achieved without prior etching of the enamel.

The concentration of the initiator system in the fissure sealant is also crucial, since a high turnover of crosslinking during polymerization also results in good adhesion to the substrate, in this case enamel. The content of the combination of camphorquinone (CQ)/tertiary amine (2-n-butoxyethyl)-4-(dimethylamino)-benzoate, BEDB) should be greater than 0.3 wt.-%/greater than 0.6 wt.-% as initiator system in the monomer component, preferably in the total composition of the fissure sealant. Table 1 shows the shear bond strength values obtained on ground bovine tooth enamel (BTE), uncut enamel (BTE-unground/BTE-uncut) after thermocycling (TWL) in otherwise qualitatively and quantitatively identically formulated fissure sealants with a filler content of 40 wt.-% (measured according to ISO 29022 in MPa). The shear bond strength could be significantly increased with the above mentioned initiator system and with increased concentration of unground BTE even after thermocycling (TWL).

Particle sizes of the fillers: The particle sizes are determined by laser diffraction. As a rule, particle size determination is carried out by laser diffraction with the Cilas instrument or alternatively Horiba LA-950 (Retsch) or DT1200 (Dispersion Technology). Unsilanized fillers are measured in water (dest) and silanized fillers are measured in isopropanol. The accuracy of the particle size determination is preferably plus/minus 0.1 micrometer for fillers with a particle size of, for example, 0.4 micrometer or d₅₀=0.4 micrometer. The accuracy for particle sizes of 1.5 micrometers or D50 1.5 micrometers is also at least in this range.

### List of Abbreviations:

| Abbreviations | |
|---|---|
| in Ex. | Chem. designation |
| UDMA | 1,6-Bis(methacryloxy-2-ethoxycarbonylamino)- |
| | 2,4,4-trimethylhexane |
| DDDMA | Dodecanediol dimethacrylate |
| MDP | 10-methacryl-oyloxydecyl phosphate |
| MMD404 | Formula I with formula III and two formulas IV |
| MMD406 | Formula I with formula IIIa/IIIb and two formulas V, |
| | n = 1 −10 |

TABLE 1
Shear bond strength data BTE 24 h and BTE unground TWL Enamel:

| | | BTE-unpolished |
|---|---|---|
| | BTE 24 h | TWL |
| 0.3 wt.-% CQ/ | 7.5 [MPa] | 18.4 [MPa] |
| 0.6 wt.-% tertiary amine | | |
| 0.6 wt.-% CQ/ | 11.3 [MPa] | 24.5 [MPa] |
| 1.2 wt.-% tert. Amine | | |

In compositions with initiator system content below 0.3 CQ/0.6 BEDB, such as. 0.1 CQ/0.2 BEDB and 0.05 CQ/0.05 BEDB, the shear bond strength to enamel is 9.0 MPa in the first case and 17.1 MPa after TWL, and in the second case with even lower concentration only 6.3 MPa (24 h) and 7.8 MPa after TWL.

The additional use of 4-META (4-methacryloyloxyethytrimellitic acid anhydride) in combination with a phosphoric acid containing monomer, such as MDP (10-methacryloyloxy decyl dihydrogen phosphate) significantly increases the melt adhesion. In particular in combination with a crosslinker/plasticizer with a longer PEG group than in TEGDMA, such as hepta (propylene glycol) diacrylate (n=7), hepta (ethylene glycol) diacrylate, (n=7), or dodecanediol di-methacrylate (DDDMA). High shear bond strengths can also be achieved with fissure sealers containing a combination of acidic monomers of carboxylic acids or carboxylic acid anhydrides with acidic esters of a phosphoric acid and two different alkylene oxide-based difunctional methacrylates comprising TEGDMA and PEGDMA (polyethylene glycol dimethylacrylate), TEGDMA and polyethylene glycol diacrylate, or TEGDMA and DDDA. Alternatively, very high shear bond strength can be achieved with fissure sealants by combining acidic monomers of carboxylic acids or carboxylic acid anhydrides with acidic esters of a phosphoric acid and two different alkylene oxide-based difunctional acrylates comprising polypropylene glycol diacrylates with n=3 to less than 15, in particular with n=3 to 9. The propylene glycol diacrylate-based fissure sealants also show reduced water absorption with simultaneously increased shear bond strength with and without TWL.

TABLE 2
Fissure sealants with A) monomer component containing
0.6 wt.-% CQ and 1.2 wt.-% BEDB, which is 100
wt.-% of the total composition.

| | | Exam- | | Exam- | | Exam- | Exam- |
|---|---|---|---|---|---|---|---|
| | VG1 | ple 1 | VG 2 | ple 2 | VG 3 | ple 3 | ple 4 |
| UDMA | 74.1 | 69.1 | 69.1 | 65.1 | 69.1 | 65.1 | 60.1 |
| TEGDMA | 14.1 | 15.1 | 14.1 | 14.1 | 14.1 | 14.1 | 14.1 |
| MDP | 10 | 10 | 10 | 10 | 10 | 10 | 10 |
| 4-META | | 4 | | 4 | | 4 | 4 |
| Polyeth- | | | 5 | 5 | | | 5 |
| ylenglycol | | | | | | | |
| dimethyl | | | | | | | |
| acrylate, | | | | | | | |
| n = 13 | | | | | | | |
| DDDMA | | | | | 5 | 5 | 5 |
| SBS BTE | 15.9 | 16.8 | 14.9 | 26.3 | 14.4 | 23.7 | 16.9 |
| [MPa], | | | | | | | |
| 24 h | | | | | | | |
| (without | | | | | | | |
| TWL) | | | | | | | |

Subsequently, fissure sealers with a monomer component containing 60 wt.-% and with a filler component content of 40 wt.-% in the total composition of 100 wt.-% were also prepared as fissure sealers. The bonding performance of the fissure sealant according to the invention clearly stands out from the competitor Constic (15 MPa fusion bond strength 24 h and 12 MPa fusion bond strength according to TWL) and Vertise Flow (16.4 MPa fusion bond strength 24 h and 2.1 MPa fusion bond strength according to TWL). These are not special fissure sealants, but self-adhesive flows that are released as fissure sealants, but are to be applied in the same way as the present invention (without a prior etching step).

TABLE 3
Fissure sealants with A) monomer component amounting to
100 wt.-% of the total composition.

| Fissure sealant | VG 4 | VG5 | VG6 | Example 5 |
|---|---|---|---|---|
| Monomer component | | | | |
| UDMA | 74.1 | 74.1 | 74.1 | 72.1 |
| TEGDMA, ethylene glycol, n = 3 | 14.1 | 14.1 | 14.1 | 12.1 |
| MMD404 | 10 | | | |
| MDP (10-methacryl-oyloxydecyl | | 10 | | |
| phosphate) | | | | |
| MMD406 | | | 10 | 10 |
| 4-META | | | | 4 |
| CQ (Champerquinone) | 0.6 | 0.6 | 0.6 | 0.6 |
| BEDB (2-n-butoxyethyl)-4- | 1.2 | 1.2 | 1.2 | 1.2 |
| (dimethylamino)-benzoate | | | | |
| Total: | 100 | 100 | 100 | 100 |
| BTE [MPa] TWL | 11.7 | 6.2 | 2.8 | 9.4 |
| BTD [MPa] TWL | 13.7 | 18.2 | 7.6 | 23 |

TABLE 4a
Fissure sealant A) 60 wt.-%, B) 40 wt.-%

| | Example 6 | Example 7 | Example 8 |
|---|---|---|---|
| Monomer component | | | |
| UDMA | 35.97 | 38.37 | 38.37 |
| TEGDMA, ethylene glycol, n = 3 | 6 | 3 | 0 |
| PEGDMA, ethylene glycol, n = 7 | 6 | 9 | 12 |
| Hepta(propylene glycol) diacrylate | | | |
| MDP (10-methacryl-oyloxydecyl | 6 | 6 | 6 |
| phosphate) | | | |
| 4-META | 4.8 | 2.4 | 2.4 |
| CQ (Champerquinone) | 0.36 | 0.36 | 0.36 |
| BEDB (2-n-butoxyethyl)-4- | 0.72 | 0.72 | 0.72 |
| (dimethylamino)-benzoate | | | |
| 2-hydroxy-4- | 0.15 | 0.15 | 0.15 |
| methoxybenzophenone | | | |
| Filler component | | | |
| Barium aluminum borofluorosilicate | 25 | 25 | 25 |
| glass, 1.5 μm, 2.3 wt.-% silanized | | | |
| Barium aluminum borofluorosilicate | 12.5 | 12.5 | 12.5 |
| glass, 0.4 μm 5.6 wt.-% silanized | | | |
| YBF (ytterbium fluoride, YBF )₃ | 2.5 | 2.5 | 2.5 |
| Total: | 100 | 100 | 100 |
| BTE [MPa] | 19.2 | 18.2 | 16.7 |
| BTE unground, TWL [MPa] | 16.9 | 17.7 | 16.9 |
| Water absorption (sol.) (μg/mm³) | | 41.5 | 44.9 |
| Hardness OS | 162 | 143 | 166 |
| Hardness US | 157 | 140 | 180 |
| Film thickness [mm] | 15 | 11 | 15 |
| Flexural strength [MPa] | 103 | 103 | 112 |
| E-modulus [MPa] | 3197 | 3109 | 3338 |

Shear bond strength on aprismatic enamel was performed after thermal cycling (SAFI without etching step/competitor with previous etching step before application) with 5000 cycles (ISO 25022). Generally described, aprismatic enamel (MZS aprismatic human tooth enamel) consists of unprepared tooth enamel that has no enamel prisms. Aprismatic enamel does not produce a retentive etching pattern.

TABLE 4b
Fissure sealants on aprismatic enamel after thermocycling
(thermocycling load).

| | | | Helioseal F | Clinpro |
|---|---|---|---|---|
| | | | 30 s | 30 s |
| | Example 9 | Example 10 | etched | etched |
| | TWL 5,000 | TWL 5,000 | TWL 5,000 | TWL 5,000 |
| | MZS | MZS | MZS | MZS |
| | aprismat. | aprismat. | aprismat. | aprismat. |
| Mean value | 14.6 | 12.8 | 13.6 | 10.1 |

Despite a prior etching step of the enamel, the fissure sealants Helioseal F and Clinpro exhibit lower or comparable shear bond strength to those according to the invention without a prior etching step.

TABLE 5
Fissure sealant A) 60 wt.-%, B) 40 wt.-%

| | Example 11 | Example 12 | Example 13 |
|---|---|---|---|
| Monomer component | | | |
| UDMA | 43.17 | 43.71 | 44.07 |
| Hepta(propyleneglycol) | 12 | 12 | 12 |
| diacrylate, n = 7 | | | |
| MDP | 2.4 | 2.4 | 2.4 |
| 4-META | 1.2 | 1.2 | 1.2 |
| 1,7,7-Trimethyl- | 0.36 | 0.18 | 0.06 |
| bicyclo[2.2.1]hepta-2,3-dion | | | |
| 2-n-Butoxyethyl-4- | 0.72 | 0.36 | 0.12 |
| (dimethylamino)benzoate | | | |
| 2-Hydroxy-4-methoxy- | 0.15 | 0.15 | 0.15 |
| benzophenone | | | |
| Filler component | | | |
| Barium aluminum borosilicate | 25 | 25 | 25 |
| 1.5 μm, silanized 2.3 wt.-% | | | |
| Barium aluminum boron | 12.5 | 12.5 | 12.5 |
| fluorosilicate 0.4 μm, | | | |
| silanized 5.6 wt.-% | | | |
| Ytterbiumfuoride | 2.5 | 2.5 | 2.5 |
| Total | 100 | 100 | 1000 |
| BTE unground, uncut, | 24.5 | 18.4 | 17.1 |
| TWL (MPa) | | | |

Unground means that the enamel has not been ground beforehand, but only cleaned, as would be the case with fissure sealing without an etching step. Consequently, the retentive anchorage option is still minimized. The bond is therefore formed via a chemical bond.

TABLE 6
Fissure sealants A) 60 wt.-%, B) 40 wt.-% with a variation of the
proportions of acidic monomer

| | Example 14 | Example 15 | Example 16 | Example 17 |
|---|---|---|---|---|
| Monomer component | | | | |
| UDMA | 38.37 | 41.37 | 43.17 | 38.91 |
| Hepta(propyleneglycol) diacrylate, | 12 | 12 | 12 | 12 |
| n = 7 | | | | |
| MDP | 6 | 3 | 2.4 | 6 |
| 4-META | 2.4 | 2.4 | 1.2 | 2.4 |
| 1,7,7-Trimethyl-bicyclo- | 0.36 | 0.36 | 0.36 | 0.18 |
| [2.2.1]hepta-2,3-dion | | | | |
| 2-n-Butoxyethyl-4- | 0.72 | 0.72 | 0.72 | 0.36 |
| (dimethylaminobenzoate | | | | |
| 2-hydroxy-4- | 0.15 | 0.15 | 0.15 | 0.15 |
| methoxybenzophenone | | | | |
| Filler component | | | | |
| Barium aluminum borosilicate | 25 | 25 | 25 | 25 |
| 1.5 μm, silanized 2.3 wt.-% | | | | |
| Barium aluminum borosilicate | 12.5 | 12.5 | 12.5 | 12.5 |
| 0.4 μm, silanized 5.6 wt .- % | | | | |
| Ytterbium fluoride | 2.5 | 2.5 | 2.5 | 2.5 |
| | 100 | 100 | 100 | 100 |
| Flexural strength [MPa] | 112 | 101 | 105 | 103 |
| E-modulus [MPa] | 3338 | 3118 | 3341 | 3460 |
| Water absorption | 44.9 | | | |
| [ g/mm ]μ³ | | | | |
| Solubility [ g/mm ]μ³ | 1.2 | | | |
| Film thickness | >6 mm | >6 mm | >6 mm | >6 mm |
| SBS BTE [MPa] | 16.7 | 15.5 | 11.3 | 14.7 |
| SBS BTE-unpolished TWL [MPa] | 16.9 | 19.4 | 24.5 | 22.6 |

TABLE 7
Change in hydrophilic alkylene oxide-based (meth)acrylates.

| | Example 18 | Example 19 | Example 20 | Example 21 | Example 22 |
|---|---|---|---|---|---|
| Monomer component | | | | | |
| UDMA | 38.37 | 38.37 | 38.37 | 38.37 | 38.37 |
| TEGDMA, ethylene glycol, n = 3 | 12 | | | | |
| DDDMA, dodecane | | 12 | | | |
| PEGDMA, ethylene glycol, n = 7 | | | 12 | | |
| Hepta(propylene glycol) | | | | | |
| diacrylate | | | | | |
| Tri(propylene glycol)- | | | | 12 | |
| diacrylate, n = 3 | | | | | |
| Hepta(propylene glycol) | | | | | 12 |
| diacrylate, n = 7 | | | | | |
| MDP | 6 | 6 | 6 | 6 | 6 |
| 4-META | 2.4 | 2.4 | 2.4 | 2.4 | 2.4 |
| 1,7,7-Trimethyl-bicyclo- | 0.36 | 0.36 | 0.36 | 0.36 | 0.36 |
| [2.2.1]hepta-2,3-dion | | | | | |
| 2-n-butoxyethyl-4-(dimethyl- | 0.72 | 0.72 | 0.72 | 0.72 | 0.72 |
| amino) benzoate | | | | | |
| 2-hydroxy-4- | 0.15 | 0.15 | 0.15 | 0.15 | 0.15 |
| methoxybenzophenone | | | | | |
| Filler | | | | | |
| Barium aluminum borosilicate | 24.5 | 24.5 | 24.5 | 25 | 25 |
| 1.5 μm, silanized 2.3 wt.-% | | | | | |
| Barium aluminum borosilicate | 12.5 | 12.5 | 12.5 | 12.5 | 12.5 |
| 0.4 μm, silanized 5.6 wt.-% | | | | | |
| Ytterbium fluoride | 3 | 3 | 3 | 2.5 | 2.5 |
| Total | 100 | 100 | 100 | 100 | 100 |
| BTE-unpolished [MPa] | 23.7 | 21.2 | 20.1 | 29 | |
| BTE-unground TWL [MPa] | 16.4 | 13.4 | 11.6 | 15.4 | 16.9 |
| Flexural strength [MPa] | 109 | 89 | 74 | 114 | 112 |
| E-modulus [MPa] | 3488 | 3105 | 1731 | 3633 | 3338 |
| Water absorption [μg/mm³] | 45.6 | 32.0 | 76.4 | 39.5 | 44.9 |
| Solubility [μg/mm³] | 0.5 | 0.0 | 1.3 | 0.7 | 1.2 |

Water absorption is lowest for a fissure sealant with DDDMA or short-chain tri (propylene glycol) diacrylate, n=3. The shear bond strength is greater than or equal to 20 MPa without thermocycling and well above 10 MPa with thermocycling for the measured examples of Table 8. The short-chain polyethylene glycol diacrylates, DDDMA and the polypropylene diacrylates show higher E-modulus compared to the fissure sealant with PEGDMA, ethylene glycol, n=7.

TABLE 8
Fissure sealants A) 60 wt.-%, B) 40 wt.-% with a
variation of acidic monomers

| | Example 23 | Example 24 |
|---|---|---|
| Monomer component | | |
| UDMA | 38.37 | 43.17 |
| Tri(propylene glycol) diacrylate | 12 | 12 |
| MDP | 6 | 2.4 |
| 4-META | 2.4 | 1.2 |
| 1,7,7-Trimethyl-bicyclo- | 0.36 | 0.36 |
| [2.2.1]hepta-2,3-dion | | |
| 2-n-butoxyethyl-4-(dimethylamino) | 0.72 | 0.72 |
| benzoate | | |
| 2-hydroxy-4-methoxybenzophenone | 0.15 | 0.15 |
| Filler component | | |
| Barium aluminum borosilicate 1.5 μm, | 25 | 25 |
| silanized 2.3 wt.-% | | |
| Barium aluminum borosilicate 0.4 μm, | 12.5 | 12 |
| silanized 5.6 wt.-% | | |
| Ytterbium fluoride | 2.5 | 3 |
| | 100 | 100 |
| Flexural strength [MPa] | 114 | 112 |
| E-modulus [MPa] | 3633 | 4009 |
| Water absorption | 39.5 | |
| Solubility [ g/mm ]μ³ | 0.7 | |
| Film thickness [ g/mm ]μ³ | 17 | |
| BTE unground | 29.0 | 22.3 |
| BTE unground TWL | 15.4 | 15.2 |

DIN-ISO 6874 (2015) specifies a curing depth of 1.5 mm for fissure sealants. With the formulations according to the invention, a curing depth of 3.4 mm (Translux wave, LED, 440-480 nm, power density greater than 1200 mmW/cm²) can be realized.

1. Self-adhesive, radiation-curable, dental fissure sealant comprising
A) a monomer component comprising
a) at least one at least difunctional urethane (meth)acrylate, wherein in each case independently one urethane (meth)acrylate is selected from urethane methacrylate
and urethane acrylate,
b) at least one hydrophilic alkylene oxide-based difunctional (meth)acrylate-based crosslinker which i) does not comprise urethane (meth)acrylate and/or ii) does not comprise aromatic residues, or a mixture of at least two of these crosslinkers, wherein in each case independently one (meth)acrylate is selected from methacrylate and acrylate, wherein the at least one hydrophilic alkylene oxide-based difunctional (meth)acrylate-based crosslinker comprises polypropylene glycol di (meth)acrylate with n=2 to n=20,
c) comprising acid monomers
i) at least one olefinic carboxylic acid and/or at least one olefinic carboxylic acid anhydride, and
ii) at least one olefinic acid ester of a phosphoric acid, and/or at least one olefinic acid ester of a thiophosphoric acid, or a mixture of i) and ii),
d) at least one initiator and/or initiator system each having an absorption maximum of 420 to 550 nm and comprising a diketone and optionally at least one co-initiator selected from a tert-amine and a 1,2 methylenedioxybenzene, and B) a filler component comprising at least one dental glass or a mixture of dental glasses,
wherein A) the monomer component and B) the filler component are present in a mass ratio of 60:40 to 100:0 in the total composition with 100 wt.-% of the fissure sealant.

2. Fissure sealant according to claim 1, wherein it comprises
A) a monomer component comprising
a) 40 to 80 wt.-% of at least one at least difunctional urethane (meth)acrylate,
b) 10 to 35 wt.-% of at least one hydrophilic alkylene oxide-based difunctional (meth)acrylate-based crosslinker which i) does not comprise urethane (meth) acrylate and/or ii) does not comprise aromatic residues, or a mixture of at least two of these crosslinkers,
c) 5 to 30 wt.-% of acidic monomers comprising
i) at least one olefinic carboxylic acid and/or at least one olefinic carboxylic acid anhydride, and
ii) at least one olefinic acid ester of a phosphoric acid, and/or at least one olefinic acid ester of a thiophosphoric acid, or a mixture of i) and ii),
d) 0.3 to 5 wt.-% of at least one initiator and/or initiator system each having an absorption maximum of 420 to 550 nm, the content of the at least one diketone is 0.3 wt.-% in the monomer component, and optionally at least one tert-amine is present as co-initiator or a 1,2-methylenedioxybenzene is present as co-initiator,
e) 0 to 0.5 wt.-% of at least one white pigment,
wherein the total composition of monomer component A) is 100 wt.-%, and B) a filler component comprising
a) 90 to 100 wt.-% of at least one dental glass or a mixture of dental glasses,
b) 0 to 10 wt.-% of inorganic fluoride, metal oxide and/or mixed oxide,
c) 0 to 0.5 wt.-% of at least one white pigment, the total composition of filler component B) being 100 wt.-%,
wherein in the fissure sealant the A) monomer component and B) filler component are present in a mass ratio of 60:40 to 100:0, and wherein the total composition of the fissure sealant is 100 wt.-%.

3. Fissure sealant according to claim 1, wherein it comprises A) the monomer component optionally in admixture with B) the filler component, wherein the fissure sealant comprises
a) 25 to 80 wt.-% of at least one at least difunctional urethane (meth)acrylate,
(b) 8 to 20 wt.-% of at least one hydrophilic alkylene oxide-based difunctional (meth)acrylate-based crosslinker which i) does not comprise urethane (meth)acrylate and/or ii) does not comprise aromatic residue, or a mixture of at least two of these crosslinkers, wherein the at least one hydrophilic alkylene oxide-based difunctional (meth)acrylate-based crosslinker comprises polypropylene glycol di (meth)acrylate with n=2 to n=20,
c) 3 to 20 wt.-% of acidic monomers comprising
i) at least one olefinic carboxylic acid and/or at least one olefinic carboxylic acid anhydride, and
ii) at least one olefinic acid ester of a phosphoric acid and/or at least one olefinic acid ester of a thiophosphoric acid, or a mixture of i) and ii),
d) 0.8 to 3 wt.-% of at least one initiator and/or initiator system, each having an absorption maximum of 420 to 550 nm and comprising a diketone and optionally at least one co-initiator selected from a tert-amine and a 1,2-methylenedioxybenzene, and B) 0 to 40 wt.-% of a filler component comprising at least one dental glass or a mixture of dental glasses, wherein the total composition of the fissure sealant is 100 wt.-%.

4. Fissure sealant according to claim 1, wherein the c) acidic monomers comprising i) at least one olefinic carboxylic acid and/or the at least one olefinic carboxylic anhydride, and ii) the at least one olefinic acidic ester of phosphoric acid, in particular a monoester of a phosphoric acid, and/or the at least one olefinic acidic ester of a thiophosphoric acid, in particular a monoester of a thiophosphoric acid, are present in a mass ratio of from 1:3 to 1:1.

5. Fissure sealant according to claim 1, wherein c) the acidic monomers comprise
i) at least one olefinic carboxylic acid and/or at least one olefinic carboxylic acid anhydride, and
ii) at least one olefinic acid ester of a phosphoric acid and/or at least one olefinic acid ester of a thiophosphoric acid, or
iii) a mixture of i) and ii), wherein i) and ii) are in a weight ratio of 1:1 to 2:1, in particular from 7:6 to 5:2, preferably from 5:4 to 5:2.

6. Fissure sealant according to claim 1, wherein the A) monomer component comprises in c) as i) 2 to 10 wt.-% of at least one olefinic carboxylic acid and/or at least one olefinic carboxylic anhydride, and as ii) 4 to 20 wt.-% of at least one olefinic acid ester of a phosphoric acid and/or at least one olefinic acid ester of a thiophosphoric acid, in particular at least one monoester of a phosphoric acid and a (meth)acrylate, monoester of a phosphoric acid and a urethane (meth)acrylate and/or at least one monoester of a thiophosphoric acid and a (meth)acrylate, with respect to the total composition of 100 wt.-% of the A) monomer component.

7. Fissure sealant according to claim 1, wherein it comprises
a) at least one at least difunctional urethane (meth)acrylate selected from difunctional urethane (meth)acrylate having a divalent alkylene group comprising difunctional urethane (meth)acrylates having a linear or branched divalent alkylene group having 3 to 20 carbon atoms, urethane dimethacrylate functionalized ethers having a linear or branched bivalent alkylene group with 3 to 20 C atoms, urethane dimethacrylate functionalized polyethers having a linear or branched bivalent alkylene group with 3 to 20 C atoms, particularly preferred is 1,6-bis (methacryloxy-2-ethoxycarbonylamino)-2,4,4-trimethylhexane (UDMA).

8. Fissure sealant according to claim 1, wherein it comprises, as b) at least one hydrophilic alkylene oxide-based difunctional (meth)acrylate-based crosslinker selected from
i) 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol dimethacrylate, tetrapropylene glycol dimethacrylate, dipropylene glycol diacrylate, tripropylene glycol diacrylate, tetrapropylene glycol diacrylate, and
ii) polyethylene glycol di(meth)acrylate and/or polypropylene glycol di(meth)acrylate or mixtures thereof, in particular with at least five ethylene glycol or propylene glycol groups, and/or
iii) 1,3-butylene glycol dimethacrylate (butanediol dimethacrylate), hexanediol dimethacrylate, octanediol dimethacrylate, decanediol dimethacrylate and/or dodecanediol dimethacrylate.

9. Fissure sealant according to claim 1, wherein b) comprises at least two different hydrophilic alkylene oxide-based difunctional (meth)acrylate-based crosslinkers selected from i) and ii) or i) and iii) or i), ii) and iii), wherein i) 1,2-ethanediol dimethacrylate, diethylene glycol dimethacrylate (DEGMA), triethylene glycol dimethacrylate (TEGDMA), tetraethylene glycol dimethacrylate, propylene glycol dimethacrylate, dipropylene glycol dimethacrylate, tripropylene glycol-dimethacrylate, tetrapropylene glycol dimethacrylate, dipropylene glycol diacrylate, tripropylene glycol diacrylate, tetrapropylene glycol diacrylate, and ii) polyethylene glycol di(meth)acrylate and/or polypropylene glycol di(meth)acrylate or mixtures thereof with at least five ethylene glycol or propylene glycol groups, and/or iii) 1,3-butylene glycol dimethacrylate, hexanediol dimethacrylate, octanediol dimethacrylate, decanediol dimethacrylate, dodecanediol dimethacrylate.

10. Fissure sealant according to claim 1, wherein c) comprises acidic monomers comprising
i) at least one olefinic carboxylic acid and/or at least one olefinic carboxylic anhydride comprising maleic acid, p-vinylbenzoic acid, 11-(meth)-acryloyloxy-1,1-undecanedicarboxylic acid (MAC-10), 1,4-di (meth)acryloyloxyethylpyromellitic acid, 6-(meth)acryloyloxyethylnaphthalene-1,2,6-tricarboxylic acid, 4-(meth)acryloyloxymethyltrimellitic acid and anhydrides thereof, 4-(meth)acryloyloxyethyltrimellitic acid and its anhydride, 4-(meth)acryloyloxybutyltrimellitic acid and its anhydride, 4-[2-hydroxy-3-(meth)acryloyloxy]butyltrimellitic acid and its anhydride, 2,3-bis (3,4-dicarboxybenzoyloxy) propyl (meth)acrylate, 2—, 3-, or 4-(meth)acryloyloxybenzoic acid, N—O-di (meth)acryloyloxytyrosine, O-(meth)acryloyloxytyrosine, N-(meth)acryloyloxytyrosine, N-(meth)acryloyloxyphenylalanine, N-(meth)acryloyl-p-aminobenzoic acid, N-(meth)acryloyl-O-aminobenzoic acid, adduct of glycidyl (meth)acrylate with N-phenylglycine or N-tolylglycine, 4-[(2-hydroxy-3-(meth)acryloyloxypropyl) amino]phthalic acid, 3- or 4-[N-methyl-N-(2-hydroxy-3-(meth)acryloyloxypropyl) amino]phthalic acid, (meth)acryloylaminosalicylic acid and (meth)acryloyloxysalicylic acid, olefinic carboxylic acids having at least two carboxyl groups and/or olefinic carboxylic anhydrides having at least three carboxyl groups, and mixtures comprising at least two of the carboxylic acids, and ii) at least one olefinic acidic monoester of a phosphoric acid and/or at least one olefinic acidic monoester of a thiophosphoric acid comprising acidic monoester of at least one phosphoric acid comprising at least two urethane (meth)acrylate groups, wherein at least two urethane (meth)acrylate groups are covalently bonded to an O atom of the phosphoric acid by means of a group having at least one trivalent C atom and optionally O atoms and form the monoester of the phosphoric acid, wherein the urethane (meth)acrylate groups are selected from urethane methacrylate and urethane acrylate groups, and wherein each urethane (meth)acrylate is independently selected from urethane methacrylate and urethane acrylate, 2-(Meth) acryloyloxyethyl acid phosphate, 2- and 3-(Meth) acryloyloxypropyl acid phosphate, 4-(Meth) acryloyloxybutylic acid phosphate, 6-(meth)acryloyloxyhexyl acid phosphate, 8-(meth)acryloyloxyoctyl acid phosphate, 10-(meth)-acryloyloxydecylic acid phosphate, 12-(meth)acryloyloxydodecyl acid phosphate, bis (2-(meth)acryloyloxyethyl acid phosphate, bis-2 or 3-(meth)acryloyloxy propylic acid phosphate, 2-(meth)acryloyloxyethyl phenylic acid phosphate, 2-(meth)acryloyl-oxyethyl-p-methoxy phenylic acid phosphate, as well as the corresponding thiophosphonates of the aforementioned (meth)acrylates, in particular wherein the (meth)acryloyl-functionalized acidic monomers are each independently selected from methylacryloyl- and acryloyl-functionalized acidic monomers, or mixtures of at least two of the aforementioned monoesters, or a mixture of i) and ii).

11. Fissure sealant according to claim 1, wherein the B) filler component comprises at least one a) dental glass or mixture of dental glasses, and b) optionally comprises at least one inorganic fluoride and/or amorphous metal oxide, mixed oxide, crystalline metal oxide, silicate, wherein a) the at least one dental glass comprises aluminosilicate glasses or fluoroaluminosilicate glasses, barium aluminosilicate, barium aluminoborosilicate, strontium silicate, strontium borosilicate, lithium silicate and/or lithium aluminosilicate, dental glasses containing ytterbium fluoride, and mixtures of at least two of the aforementioned dental glasses, and b) comprises ytterbium fluoride, amorphous spherical fillers based on oxide or mixed oxide, such as amorphous SiO₂, ZrO₂ or also mixed oxides of SiO₂ and ZrO₂, quartz, feldspar and mixtures of at least two of the fillers.

12. Fissure sealant according to claim 1, wherein d) the diketone of the at least one initiator and/or the at least one initiator system comprises 1,7,7-trimethyl-bicyclo-[2.2.1]-hepta-2,3-dione (champherquinone), phenylpropanedione, benzoyltrimethylgerman (BTMGe) and/or dibenzoyldiethylgerman (DBDEGe) and at least one co-initiator comprising as tert-amine 2-n-butoxyethyl-4-(dimethylamino)benzoate and/or as 1,2-methylenedioxybenzene, piperonyl alcohol (1,2-methylenedioxybenzene-4-methanol).

13. Fissure sealant according to claim 1, wherein the A) Monomer component comprises e) 0.3 to 2.5 wt.-% of at least one diketone, in particular 1,7,7-trimethyl-bicyclo-[2.2.1]-hepta-2,3-dione (champherquinone), and 0.6 to 2.5 wt.-% of at least one tert-amine, in particular 2-n-butoxyethyl-4-(dimethylamino)benzoate, in particular wherein the mass ratio diketone to co-initiator is from 1:1.5 to 1:3, preferably 1:2 with +/−0.15, with respect to the total composition of 100 wt.-% of the A) monomer component.

14. Fissure sealant according to claim 1, wherein the c) acidic monomers comprise
i) 4-methacryloyloxyethyltrimellitic acid (4-MET) and/or its anhydride (4-META), and
ii) acidic monoester of at least one phosphoric acid comprising at least two urethane (meth)acrylate groups, in particular wherein at least two urethane (meth)acrylate groups are covalently bonded to an O-atom by means of a group having at least one trivalent C-atom and optionally O atoms, are covalently bonded to an O atom of the phosphoric acid and form the monoester of the phosphoric acid, wherein the urethane (meth)acrylate groups are independently selected from urethane methyl acrylate and urethane acrylate groups, 2-methacryloyloxy ethylphenylic acid phosphate, 10-meth-acryloyloxydecylic acid phosphate, 2-acryloyloxyethylphenylic acid phosphate and/or 10-acryloyloxydecylic acid phosphate or a mixture of i) and ii).

15. Fissure sealant according to claim 1, wherein the ii) comprises at least one acidic monoester of at least one phosphoric acid of the general formulas I and/or II or mixtures of at least two of the monoesters selected from the formulas I and II 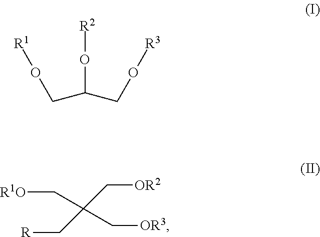
with R=CH₃, OR¹, OR² or OR³
with R¹, R² and R³ are selected from Formulas III, IIa, IIIb, IV, V, VIa, VIb, VIIa and VIIb with 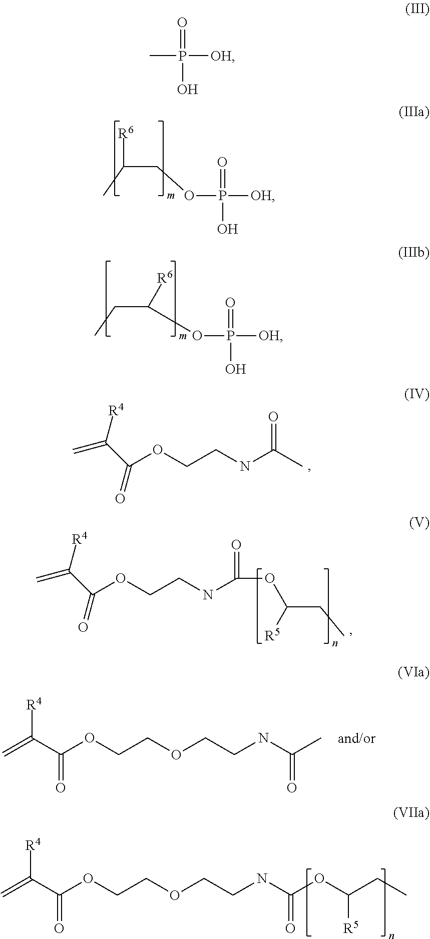
with, in formulas V and VIIa, n is independently from 1 to 100, and
wherein at least one or two residues selected from R¹, R² and R³ correspond to a group of formula III and the remaining residue or residues of R¹, R² and R³ are selected from a group of formula IV, V, VIa and/or VIIa,
wherein R⁴ and R⁵ are each independently selected from H, methyl and ethyl, in particular the remaining groups are selected from at least two formulas of IV, V, VIa and/or VIIa, wherein R⁶ is each independently selected in the groups of formulas IIIa and IIIb is selected from H and methyl, and wherein m=0 or 1 to 100 in formulas IIIa and/or IIIb, in particular 1 to 20

16. Fissure sealant according to claim 1, wherein at least one hydrophilic alkylene oxide-based difunctional (meth)acrylate-based crosslinker is selected from polyethylene glycol di (meth)acrylate and/or polypropylene glycol di(meth)acrylate or mixtures thereof.

17. Fissure sealant according to claim 1, wherein the hydrophilic alkylene oxide-based difunctional (meth)acrylate-based crosslinker comprises polyethylene glycol di (meth)acrylates, in particular TEGDMA, and polypropylene glycol di(meth)acrylates with n=2 to n=20.

18. Fissure sealant according to claim 1, wherein A) the monomer component is optionally present in admixture with B) the filler component, wherein the fissure sealant comprises
a) 30 to 80 wt.-% of at least one at least difunctional urethane (meth)acrylate,
b) 8 to 20 wt.-% of at least one hydrophilic alkylene oxide-based di-functional (meth)acrylate-based crosslinker comprising i) no urethane (meth)acrylate and/or ii) no aromatic residues, or a mixture of at least two of these crosslinkers,
c) i) 2 to 10 wt.-% of at least one olefinic carboxylic acid and/or at least one olefinic carboxylic anhydride, in particular at least one carboxylic acid-functionalized and/or carboxylic anhydride-functionalized (meth)acrylate monomer, and
ii) 4 to 20 wt.-% of at least one olefinic acid ester of a phosphoric acid and/or at least one olefinic acidic ester of a thiophosphoric acid, in particular at least one monoester of a phosphoric acid and of a (meth)acrylate, monoester of a phosphoric acid and of a urethane (meth)acrylate and/or at least one monoester of a thiophosphoric acid and of a (meth)acrylate, or a mixture of i) and ii),
d) 0.3 to 2.5 wt.-% of at least one diketone selected from 1,7,7-trimethyl-bicyclo-[2.2.1]-hepta-2,3-dione (champherquinone), phenylpropanedione, and 0.6 to 2.5 wt.-% of at least one co-initiator selected from tert-amine and 1,2-methylenedioxybenzenes, in particular selected from 2-n-butoxyethyl-4-(dimethylamino)benzoate and/or piperonyl alcohol, the mass ratio of diketone to co-initiator selected from tert-amine or 1,2-methylenedioxybenzenes being from 1:1.5 to 1:3,
e) 0 to 1 wt.-% of at least one pigment comprising titanium dioxide, and 0 to 2 wt.-% of dye, UV stabilizer and/or Vis stabilizer, and
f) 0 to 30 wt.-% of at least one dental glass,
g) 0 to 10 wt.-% of inorganic fluoride, such as ytterbium fluoride, metal oxide, mixed oxides and/or silicates, and
h) 0 to 1 wt.-%, in particular 0.1 to 0.5 wt.-%, of at least one pigment comprising titanium dioxide, and 0 to 2 wt.-% of dye, UV and/or vis stabilizer, and/or mixtures of at least two of the fillers e) to h), the above components a) to h) giving the total composition of 100 wt.-% of the fissure sealant.

19. Polymerized dental fissure sealant obtainable by polymerization of a fissure sealant according to claim 1, in particular having a shear bond strength to untreated bovine tooth enamel, in particular ground bovine tooth enamel of greater than or equal to 19 MPa, preferably greater than or equal to 22 MPa, particularly preferably greater than or equal to 23 MPa.

20. Self-adhesive, radiation-curable, dental fissure sealant or self-adhesive, polymerized, in particular radiation-cured, fissure sealant according to claim 1 for use in sealing fissures in enamel and/or dentin, and/or for use for radiation-curing, self-adhesive adhesion to substrates, to dentin and/or enamel of teeth, in particular human or veterinary teeth, and/or as an underfilling material.
